(19) United States
(12) Patent Application Publication
(10) Pub. No.: US 2008/0051317 A1
(43) Pub. Date: Feb. 28, 2008

(54) Polypeptides comprising unnatural amino acids, methods for their production and uses therefor

(76) Inventors: George Church, Brookline, MA (US); David Liu, Lexington, MA (US)

Correspondence Address: WOLF GREENFIELD & SACKS, P.C., 600 ATLANTIC AVENUE, BOSTON, MA, 02210-2206, US

(21) Appl. No.: 11/639,541

(22) Filed: Dec. 15, 2006

Related U.S. Application Data
(60) Provisional application No. 60/751,397, filed on Dec. 15, 2005.

(51) Int. Cl. A61K 38/02; C07K 2/00; C12N 5/06; C12P 21/00

(52) U.S. Cl. 514/2; 435/325; 435/71.1; 530/300

(57) ABSTRACT

Certain aspects of the present invention provide artificial polypeptides containing one or more unnatural amino acids and methods for producing and using the artificial polypeptides.

### RELATED APPLICATIONS

This application claims priority under 35 U.S.C. § 119(e) to U.S. provisional application Ser. No. 60/751,397, entitled “POLYPEPTIDES COMPRISING UNNATURAL AMINO ACIDS, METHODS FOR THEIR PRODUCTION AND USES THEREFOR,” filed on Dec. 15, 2005, the entire contents of which are incorporated herein by reference.

### BACKGROUND

Recombinant polypeptides are routinely produced in host cells and in vitro expression systems. In addition, certain methods for introducing unnatural amino acids or modifying amino acid side chains in polypeptides are known in the art. However, methods for producing polypeptides containing unnatural amino acids remain cumbersome, expensive, and inefficient.

### SUMMARY OF THE INVENTION

Aspects of the invention relate to methods for genetically modifying cells and organisms to improve their effectiveness as hosts for the expression of polypeptides incorporating one or more unnatural amino acids. Aspects of the invention also relate to genetically modified host cells and organisms, and methods for producing artificial polypeptides containing one or more unnatural amino acids. Other aspects of the invention relate to polypeptides comprising one or more unnatural amino acids and methods for their production. In particular, artificial polypeptides of the invention may contain one or more unnatural amino acid side chains that do not react (or react poorly) with most or all biological molecules under normal physiological conditions, but that can react efficiently with other unnatural groups to form non-native bonds (e.g., non-native intramolecular cross-links within proteins or non-native intermolecular cross-links between proteins and other compounds). Examples of chemical reactions that don't interfere with biological or intracellular chemistry include certain cycloaddition, condensation, and nucleophilic reactions between unnatural amino acid side chains. The term “unnatural amino acid” refers to any amino acid, modified amino acid, and/or amino acid analogue that is not one of the 20 naturally occurring amino acids or seleno cysteine.

In one aspect, methods of the invention are useful for producing large amounts of modified polypeptides that contain at least one unnatural amino acid. In certain embodiments, modified polypeptides may be useful for medical purposes. For example, a modified polypeptide may be a therapeutic protein containing at least one unnatural amino acid that confers a desirable functional or structural property on the protein. In other embodiments, modified polypeptides may be useful for a range of different applications. For example, a modified polypeptide containing one or more unnatural amino acids may be useful for agricultural, environmental, industrial, nutritional, forensic, research, and/or other applications. The term “polypeptide”, and the terms “protein” and “peptide” which are used interchangeably herein, refers to a polymer of amino acids, including, for example, gene products, naturally-occurring proteins, homologs, orthologs, paralogs, fragments, and other equivalents, variants and analogs of the foregoing.

In one aspect, a host cell may be modified to improve its artificial polypeptide production efficiency by reducing the number of copies of a predetermined codon in one or more open reading frames on the host genome. The predetermined codon can be reassigned for recognition by a modified tRNA that is charged with an unnatural amino acid. Accordingly, the predetermined codon can be introduced into an open reading frame on a template nucleic acid at one or more positions at which an unnatural amino acid is to be incorporated during synthesis of the encoded polypeptide, thereby generating an artificial polypeptide containing one or more unnatural amino acids. It should be appreciated that a host organism can be modified to reduce the number of two or more codons so that they can be reassigned to be recognized by two or more different modified tRNAs charged with two or more different unnatural amino acids. In one embodiment, all of the copies of one or more predetermined codons are removed from the open reading frames on the genome (e.g., replaced by alternative codons). Aspects of the invention also provide methods for making large scale genetic changes throughout the genome of an organism (e.g., using hierarchical or sequential genetic replacement methods described herein) in order to remove (e.g., replace) certain codons throughout the genome. It should be understood that the term “genome” refers to the whole hereditary information of an organism that is encoded in the DNA (or RNA for certain viral species) including both coding and non-coding sequences. In various embodiments, the term may include the chromosomal DNA of an organism and/or DNA that is contained in an organelle such as, for example, the mitochondria or chloroplasts. The term “mitochondrial genome” refers to the genetic material contained in the mitochondria and the term “chloroplast genome” refers to the genetic material contained in the chloroplast.

In one aspect, the invention provides a method of expressing an artificial polypeptide comprising at least one unnatural amino acid by exposing a host cell to an expression condition wherein a first modified tRNA charged with a first unnatural amino acid is available for protein synthesis. The host cell may include a genome that is modified to replace at least a threshold number of copies of a first codon with one or more first alternative codons, wherein the first modified tRNA recognizes the first codon with greater specificity than any of the one or more first alternative codons. The host cell also may include a first nucleic acid comprising an open reading frame having at least one copy of the first codon, wherein translation of the open reading frame results in expression of an artificial polypeptide comprising at least one unnatural amino acid inserted at a position in the polypeptide that corresponds to the position of the first codon in the open reading frame. In some embodiments, an artificial polypeptide may be isolated from a host cell preparation of the invention.

In some embodiments, a first unnatural amino acid may form a non-native bond (e.g., a non-native covalent bond) with a second amino acid inside the host cell. The second amino acid may be a second unnatural amino acid. As used herein, a “non-native” bond involving a reactive group that is not found in biological systems in nature. For example, a non-native bond may link two unnatural amino acid side chains. In other examples, a non-native bond may link an unnatural amino acid side chain to a natural biomolecule (e.g., via a natural amino acid side chain). For example, a non-native bond may be formed via a pericyclic reaction, a condensation, or nucleophilic addition. However, other types of reactions also may be used to form non-native bonds. Certain reactions may proceed spontaneously under biological conditions. Other reaction may require the addition of a catalyst (e.g., a metal ion) or exposure to appropriate pH, temperature, salt, or other conditions. In certain embodiments, one or more of the unnatural amino acids (e.g., all of the unnatural amino acids) in an artificial polypeptide of the invention contain a bioorthogonal side chain attached to the alpha carbon of the amino acid. As used herein, the term “bioorthogonal” refers to non-biological functional groups that, under normal biological conditions, are stable and inert to biological functional groups typically found in biological systems. Under certain circumstances, two bioorthogonal groups within a single molecule may interact with each other to form a non-native intramolecular covalent bond. Similarly, two bioorthogonal groups in different molecules may interact with each other to form a non-native intermolecular covalent bond. In some embodiments, a non-native intramolecular covalent bond may stabilize a polypeptide. In other embodiments, a non-native intermolecular covalent bond may connect two polypeptides. In further embodiments, a non-native intermolecular covalent bond may connect a polypeptide to a non-peptide molecule (e.g., to another biological molecule or to a non-biological molecule).

In certain embodiments, the formation of a non-native bond may be promoted during production of an artificial polypeptide by including a step of exposing the artificial polypeptide to a compound that reacts with a first unnatural amino acid. The polypeptide may be exposed to the compound after the polypeptide is isolated from the host cell. Any suitable reactive compound may be used, for example PEG, a monoalkyl hydrocarbon, a dialkyl hydrocarbon, a monosaccharide, a polysaccharide, or a combination thereof. However, other reactive compound(s) may be used as the invention is not limited in this respect. Accordingly, in some embodiments the artificial polypeptide may be PEGylated, alkylated, glycosylated, carboxymethylated, or a combination thereof.

In certain embodiments, an artificial polypeptide may be isolated from a suitable preparation of material from a host cell expression system. The preparation may be a crude cell lysate, a cell culture supernatant, a partially purified preparation of the artificial protein, or any other suitable preparation.

Accordingly, one aspect of the invention provides an artificial polypeptide that contains at least one unnatural amino acid. The artificial polypeptide may be provided in a suitable composition or in a pharmaceutical preparation. Alternatively, the artificial polypeptide may be dried (e.g., lyophilized) and provided as a dry powder. Aspects of the invention are useful for promoting the expression of large amounts (e.g., industrial scale amounts) of an artificial polypeptide. Therefore, compositions or preparations of the invention may include large amounts (e.g., therapeutic amounts or commercial amounts) of the artificial polypeptide. The presence of at least one unnatural amino acid in the artificial polypeptide may enhance structural and or functional properties of the artificial polypeptide. In some embodiments, an artificial polypeptide may be a stabilized protein such as a stabilized therapeutic protein with an increased storage time and/or an increased circulatory half-life after it is administered to a patient.

Another aspect of the invention relates to a host cell including a genome that is modified to replace at least a threshold number of copies of a first codon with one or more first alternative codons. The host cell also may include a first nucleic acid that comprises an open reading frame encoding an artificial polypeptide, wherein the open reading frame comprises at least one copy of the first codon, and wherein the first codon is recognized by a first modified tRNA with greater specificity than any of the one or more alternative codons. In one embodiment, the first stop codon is UAA or UAG, the alternative stop codon is UGA, and the host cell is a prokaryotic cell with a genome comprising a mutation that reduces expression of release factor 1. In another embodiment, the first stop codon is UAA or UGA, the alternative stop codon is UAG, and the host cell is a prokaryotic cell with a genome comprising a mutation that reduces expression of release factor 2. The mutation may be a deletion, a point mutation, or any other form of genetic rearrangement that reduces (or abolishes) expression of the appropriate release factor.

In some embodiments, the host cell also may comprise a second nucleic acid that encodes the first modified tRNA, a third nucleic acid that encodes a first modified tRNA synthetase that promotes charging (amino-acylation) of the first modified tRNA with the first unnatural amino acid, or a combination thereof. It should be appreciated that the first, second, and third nucleic acids each may be, independently, an extra-chromosomal nucleic acid, a plasmid, a nucleic acid integrated into a viral genome, part of a genomic nucleic acid of the host cell, or any other suitable nucleic acid. In some embodiments, at least two of the first, second, and third nucleic acids are genomic nucleic acids in the host cell.

A further aspect of the invention provides a nucleic acid comprising an open reading frame encoding an artificial polypeptide. The open reading frame may include a first codon that is recognized by a first modified tRNA charged with a first unnatural amino acid. In some embodiments, the first codon is at a position selected in the open reading frame such that an unnatural side chain of the first unnatural amino acid forms an internal covalent bond with a side chain of a second amino acid in a folded structure of the encoded artificial polypeptide. In certain embodiments, the open reading frame also may include a second codon that is different from the first codon, and that is recognized by a second modified tRNA charged with a second unnatural amino acid. In some embodiments, the first codon may be a stop codon and the open reading frame may be terminated by an alternative stop codon. In certain embodiments, the second codon also may be a stop codon and the open reading frame may be terminated by an alternative stop codon that is different from the first and second codons.

In certain embodiments, a nucleic acid may encode an artificial polypeptide that forms a secondary structural motif (e.g., an alpha helix, a beta sheet, or other secondary structural motif). A nucleic acid of the invention may be provided in the form of a nucleic acid cassette that may be introduced into an open reading frame to provide a sequence encoding an unnatural amino acid. In certain embodiments, the nucleic acid may be provided on a vector. Any of the nucleic acids described herein may be provided in a host cell. As used herein, the term “nucleic acid” refers to polynucleotides such as deoxyribonucleic acid (DNA), and, where appropriate, ribonucleic acid (RNA). The term should also be understood to include analogs of either RNA or DNA made from nucleotide analogs (including analogs with respect to the base and/or the backbone, for example, peptide nucleic acids, locked nucleic acids, mannitol nucleic acids, etc.), and, as applicable to the embodiment being described, single-stranded (such as sense or antisense), double-stranded or higher order polynucleotides.

In another aspect, the invention provides a method of stabilizing a protein by introducing a stabilized secondary structural motif into the protein, wherein the stabilized secondary structural motif comprises a first unnatural amino acid with a side chain (e.g., an unnatural bioorthogonal side-chain group) that is covalently bound the side chain of a second amino acid in the secondary structural motif. The protein may be a therapeutic protein.

In another aspect, the invention provides a method of making an expression construct for expressing a stabilized protein by introducing a nucleic acid cassette described herein into an open reading frame encoding a natural or recombinant protein. In some embodiments, a cassette encoding an artificial secondary structure containing an unnatural amino acid may be used to replace a sequence encoding a secondary structural motif in the natural or recombinant protein.

In another aspect, the invention provides a method of activating a biochemical pathway by exposing a cell to an artificial polypeptide or protein that is a modified form of a natural or recombinant protein activator of the biochemical pathway, wherein the modified form of the protein contains an unnatural amino acid (e.g., an amino acid with an unnatural bioorthogonal side chain).

In another aspect, the invention provides a method of inactivating a biochemical pathway, by exposing a cell to an artificial polypeptide or protein that is a modified form of a natural or recombinant protein is an inhibitor of the biochemical pathway, wherein the modified form of the protein contains an unnatural amino acid (e.g., an amino acid with an unnatural bioorthogonal side chain).

In a further aspect, the invention provides a method of treating a patient by administering a therapeutically effective amount of an artificial polypeptide or protein to a patient in need thereof, wherein the artificial polypeptide or protein is a modified form of a natural or recombinant therapeutic polypeptide or protein, and wherein the modified form contains an unnatural amino acid (e.g., an amino acid with an unnatural bioorthogonal side chain). In some embodiments, the artificial polypeptide or protein is therapeutically effective at a lower amount than the corresponding natural or recombinant therapeutic protein. In some embodiments, the corresponding natural or recombinant protein is an antibody, a hormone, an enzyme, a receptor, an antibiotic, a ligand, antigen, or other structural or functional protein. In certain embodiments, the patient may have a condition or disorder (e.g., an infection, cancer, diabetes, a neurodegenerative disorder, an immune system disorder, or other disease or condition).

In any aspects or embodiments relating to a host cell described herein, the host cell may be a bacterial cell (e.g., an E. coli cell), a yeast cell, an insect cell, a mammalian cell, a plant cell, or any other suitable prokaryotic or eukaryotic cell. A host cell may be grown in suspension or in a fermentor or under any other appropriate conditions. Expression of a polypeptide of interest may occur when the host cell is in a stationary growth phase. The host cell may be exposed to a condition that promotes expression of the polypeptide when the host cell reaches a stationary growth phase. However, the expression condition may include a growth medium and the artificial polypeptide may be expressed during growth of the host cell. The expression condition may involve an agent that promotes expression of the artificial polypeptide. The agent may promote transcription of a region of the first nucleic acid that includes the open reading frame. In some embodiments, the host cell may be incubated in a medium comprising a first unnatural amino acid. In some embodiments, the host cell may be incubated in a medium comprising the first modified tRNA charged with the first unnatural amino acid (or two or more modified tRNAs charged with different unnatural amino acids, etc.). In some embodiments, the host cell may have a genome that is modified to replace a first threshold number of a first stop codon and a second threshold number of a second stop codon with a third stop codon. In certain embodiments, at least half of the first codons on the genome of the host cell are replaced with one or more alternative codons. In some embodiments, all of the first codons on the host cell genome are replaced with one or more alternative codons.

In any aspects or embodiments described herein, an open reading frame encoding an artificial polypeptide or protein may include at least one copy of a second codon that is recognized by a second modified tRNA charged with a second unnatural amino acid, wherein the second unnatural amino acid is different from the first unnatural amino acid. In some embodiments, the open reading frame may include at least one copy of each of three or more different codons, wherein each codon is recognized by a different modified tRNA charged with a different unnatural amino acid. In some embodiments, the genome of the host cell may be modified to replace at least a threshold number of copies of the second codon with one or more second alternative codons, wherein the second modified tRNA recognizes the second codon with greater specificity than any of the one or more second alternative codons.

In any aspects or embodiments described herein, the first codon may be one of several degenerate codons for a natural amino acid. The term “degenerate codons” refers to two or more codons that encode for the same amino acid or a translational stop. For example, WUA, UUG, CUU, CUC, CUA and CUG are degenerate codons that encode for the amino acid leucine. Similarly, UAA, UAG and UGA are degenerate codons that signal a translational stop (e.g., “stop codons”). Accordingly, the first codon may be a first stop codon (e.g., UAA, UGA, or UAG). If the expression system is designed such that the codon selected to be recognized by a modified tRNA is a stop codon, the open reading frame should be terminated by a different stop codon to prevent translation extending through the intended termination position. For example, if UAA is recognized by the modified tRNA, the open reading frame may be terminated by a UAG or UGA stop codon. In some embodiments, at least half of the first stop codons on the genome of the host cell may be replaced with a second stop codon. In certain embodiments, all of the first stop codons on the genome of the host cell are replaced with a second stop codon. For example, in one embodiment, at least half of the UAA stop codons on the genome may be replaced with a UAG or UGA stop codon.

When selecting one or more codons to be used for introducing unnatural amino acid(s) into a predetermined polypeptide expressed in a host organism, it may be preferable to use a relatively rare codon so that it is easier and/or less disruptive to remove or replace genomic copies of the codon in the host.

In certain aspects, the invention provides host cell lines capable of expressing one or more artificial polypeptides or proteins, methods of storing the host cell lines (e.g., frozen, dried, etc.), and methods of growing the host cell lines (e.g., in liquid culture).

In some aspects, the invention relates to importing one or more host cells, host cell lines, cell lysates, polypeptide preparations, artificial polypeptides, artificial proteins, nucleic acid cassettes, pharmaceutical preparations, or any combination of two or more thereof into the United States of America.

In certain aspects, the invention relates to business methods that involve obtaining or preparing and collaboratively or independently marketing one or more host cells, host cell lines, cell lysates, polypeptide preparations, artificial polypeptides, artificial proteins, nucleic acid cassettes, pharmaceutical preparations, or any combination of two or more thereof.

In any of the aspects or embodiments described herein, any of the first nucleic acid, second nucleic acid, third nucleic acid, or any combination thereof may be a DNA molecule. In certain embodiments, one or more of these nucleic acids may be RNA, PNA, or other form of nucleic acid.

Other features and advantages of the invention will be apparent from the following detailed description, and from the claims. The claims provided below are hereby incorporated into this section by reference.

### BRIEF DESCRIPTION OF THE FIGURES

FIG. 1. Part A shows relative numbers of codons in E. coli. Part B provides an example of E. coli codons that may be freed up.

FIG. 2. Shows four examples of methods for forming non-native covalent bonds involving an unnatural amino acid side chain in a polypeptide. Part A illustrates the cycloaddition of an azide to a cycloalkyne to form a triazole ring. Part B shows the condensation of a ketone or aldehyde with a hydrazide to form a substituted hydrazone. Part C illustrates the copper-catalyzed cycloaddition of an azide to a terminal alkyne to form a triazole ring. Part D shows the condensation of a ketone or aldehyde with an aminooxy group to form an oxime ether.

FIG. 3. Simplified illustration of an example DNA molecule to be synthesized.

FIG. 4. Illustrates a microarray used in the synthesis of the exemplary DNA molecule of FIG. 3.

FIG. 5. Removal of error sequences using mismatch binding proteins.

FIG. 6. Neutralization of error sequences with mismatch recognition proteins.

FIG. 7. Strand-specific error correction.

FIG. 8. Local removal of DNA on both strands at the site of a mismatch.

FIG. 9. Another scheme for local removal of DNA on both strands at the site of a mismatch.

FIG. 10. Summarizes the effects of the methods of FIG. 8 (or equivalently, FIG. 9) applied to two DNA duplexes, each containing a single base (mismatch) error.

FIG. 11. Shows an example of semi-selective removal of mismatch-containing segments.

FIG. 12. Shows a procedure for reducing correlated errors in synthesized DNA.

FIG. 13. Shows an illustration of a method for hierarchical assembly of a synthetic genome. Part A shows the starting materials including a cell with an initial genome (empty) and variety of polynucleotide constructs (hatched; labeled A, B, C, and D). Part B illustrates intermediates having a single polynucleotide segment integrated into the host genome. Part C shows the product of conjugation followed by homologous recombination to produce intermediates having two synthetic polynucleotide constructs integrated into the genome. Part D illustrates the final synthetic genome produced by further rounds of conjugation and homologous recombination.

### DETAILED DESCRIPTION OF THE INVENTION

Methods and compositions of the invention relate to the production of artificial polypeptides containing one or more unnatural amino acids. Aspects of the invention include expression systems involving modified tRNA molecules that are charged with unnatural amino acid(s), purification methods, host cells, nucleic acids, pharmaceutical preparations, therapeutic applications, stabilized proteins, and other embodiments related to artificial polypeptides containing one or more unnatural amino acids.

Aspects of the invention provide methods for enhancing tRNA-mediated incorporation of one or more unnatural amino acids into a selected polypeptide during synthesis in an in vivo or in vitro translation system by reducing incorporation of the unnatural amino acid(s) into other polypeptides that also are being synthesized in the translation system. According to the invention, an artificial polypeptide containing an unnatural amino acid at one or more predetermined positions may be expressed from a template open reading frame that contains or is modified to contain, at predetermined positions, one or more predetermined codons that are recognized by a modified tRNA charged with an unnatural amino acid.

In one aspect, the invention provides methods for modifying a host cell to improve its ability to incorporate unnatural amino acids into a polypeptide that is expressed in the host (e.g., translated from an open reading frame on a predetermined template in the host). A template encoding an artificial polypeptide may be a nucleic acid that includes one or more predetermined codons recognized by modified tRNA(s) charged with unnatural amino acid(s). The genome of the host cell may be modified to reduce the content of the predetermined codon(s) in genomic open reading frames (e.g., in the open reading frames that do not encode the artificial polypeptide). This reduces or prevents unwanted incorporation of the unnatural amino acid(s) into polypeptides other than the artificial polypeptide of interest. In certain embodiments, the reduction of unwanted incorporation of unnatural amino acid(s) promotes improved incorporation of unnatural amino acids into the artificial polypeptide. Improved incorporation may be attributed to several factors. The reduction of unwanted incorporation results in higher amounts of modified tRNA charged with unnatural amino acid(s) available for incorporation into the artificial polypeptide(s). The reduction of unwanted incorporation also results in higher specificity of unnatural amino acid incorporation into the polypeptide(s) of interest. This may be helpful for subsequent isolation procedures and yield preparations with a higher purity of an artificial polypeptide containing unnatural amino acid(s). Accordingly, incorporation of unnatural amino acid(s) into a predetermined polypeptide may be enhanced in an expression system that contains few or no competing open reading frames including the predetermined codon. Aspects of the invention may be used in protein expression systems in vivo in a host cell or in vitro in an artificial translation or transcription/translation system. However, the reduction of unwanted incorporation may be particularly desirable in an in vivo host expression system (e.g., in a host cell) that generally contains higher levels of different nucleic acids with different open reading frames than an in vitro protein expression system. Also, the reduction of unwanted incorporation may be beneficial to an in vivo host expression system where misincorporation of unnatural amino acids into many different polypeptides may be harmful to the cell and reduce its growth and/or protein expression levels. This may be particularly important if the modified tRNA recognizes a stop codon. In this embodiment, unnatural amino acids may be added at the ends of natural polypeptides resulting not only in the incorporation of inappropriate amino acids but also inappropriate extension of cellular proteins.

In certain embodiments, an artificial polypeptide expression system of the invention includes (i) a modified tRNA that is charged with an unnatural amino acid, wherein the tRNA recognizes a predetermined codon, and (ii) a template nucleic acid that includes an open reading frame encoding a polypeptide of interest, wherein the open reading frame includes one or more copies of the predetermined codon at predetermined position(s) at which the unnatural amino acid will be incorporated into the artificial polypeptide. These elements of the expression system may be introduced into a host cell as described herein. In some embodiments, the host cell is modified as described herein to reduce or remove host codons that may compete with the predetermined codon on the template nucleic acid for recognition by the modified tRNA charged with the unnatural amino acid.

In certain embodiments, a modified tRNA is a tRNA charged with an unnatural amino acid, but for which the natural codon recognition has not been changed. In other embodiments, a modified tRNA is charged with an unnatural amino acid and also is modified to recognize a different codon (e.g., it is an nonsense suppressor tRNA that recognizes a stop codon). A modified tRNA may be provided to the growth medium of a host cell. In some embodiments, the modified tRNA is expressed in the host cell and charged with the unnatural amino acid inside the host cell in a reaction catalyzed by a tRNA synthetase. The tRNA synthetase may be a modified tRNA synthetase that specifically catalyzes the amino acylation of a particular tRNA molecule with the unnatural amino acid. In these embodiments, the host cell may be grown in the presence of the unnatural amino acid (and optionally in the presence of reduced levels, or the absence, of competing natural amino acid(s)) and the amino acylation occurs inside the host cell. Methods for producing or designing a tRNA that (1) is charged (amino-acylated) with a selected unnatural amino acid and (2) specifically recognizes one or more selected codons are known in the art and described herein (e.g., in Example 1). Similarly, methods for designing or generating modified tRNA synthetases with altered substrate recognition are known in the art and described herein (e.g., in Example 1).

In certain embodiments, the template nucleic acid is an extrachromosomal nucleic acid (e.g., a plasmid, viral genome, or other vector). However, the template nucleic acid may be integrated into the host genome. The template nucleic acid may be a DNA molecule. However, in certain embodiments, the template nucleic acid may be an RNA molecule. In some embodiments, a template DNA may include one or more promoter, enhancer, and/or other transcriptional regulatory elements (including inducible elements) to control and/or promote transcription of an RNA molecule containing the open reading frame. An RNA template or an RNA molecule transcribed from a DNA template may include one or more appropriate translational control sequences.

In certain embodiments, the open reading frame on the template nucleic acid may encode a polypeptide that is foreign to the host cell (e.g., a mammalian polypeptide expressed in a bacterial cell). In some embodiments, the polypeptide sequence may be entirely or partially artificial. In some embodiments, the polypeptide may be a host polypeptide expressed from the host genome. In any of these embodiments, the template may be modified to introduce a predetermined codon at a position in the open reading frame at which incorporation of an unnatural amino acid into the polypeptide is desired. The open reading frame also may be modified to remove the predetermined codon from any positions other than those at which unnatural amino acid incorporation is desired. In other embodiments, an open reading frame already may include appropriate codons at predetermined positions of interest. In this case, no modification to the open reading frame may be required. However, if appropriate, the open reading frame may be modified to remove copies of the predetermined codon from positions other than those of interest.

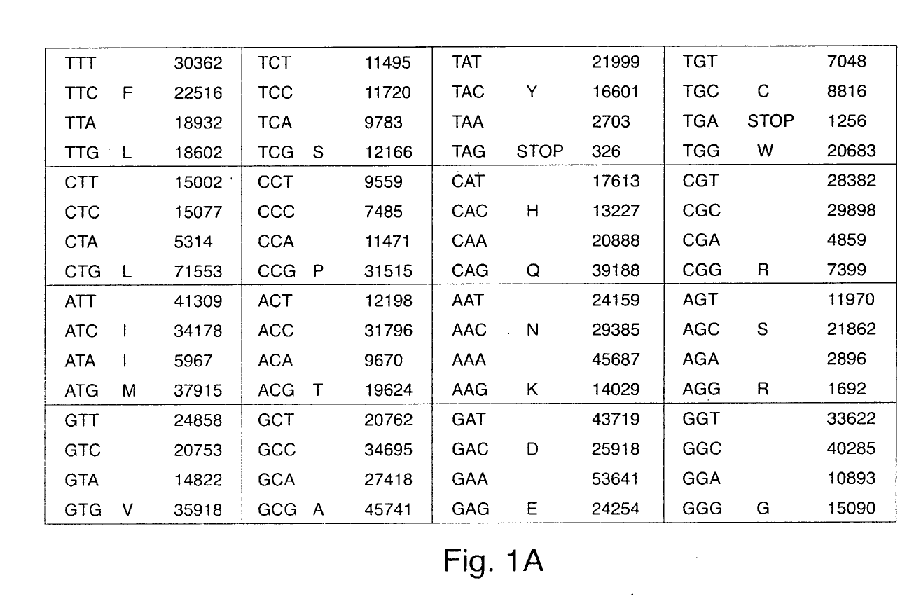

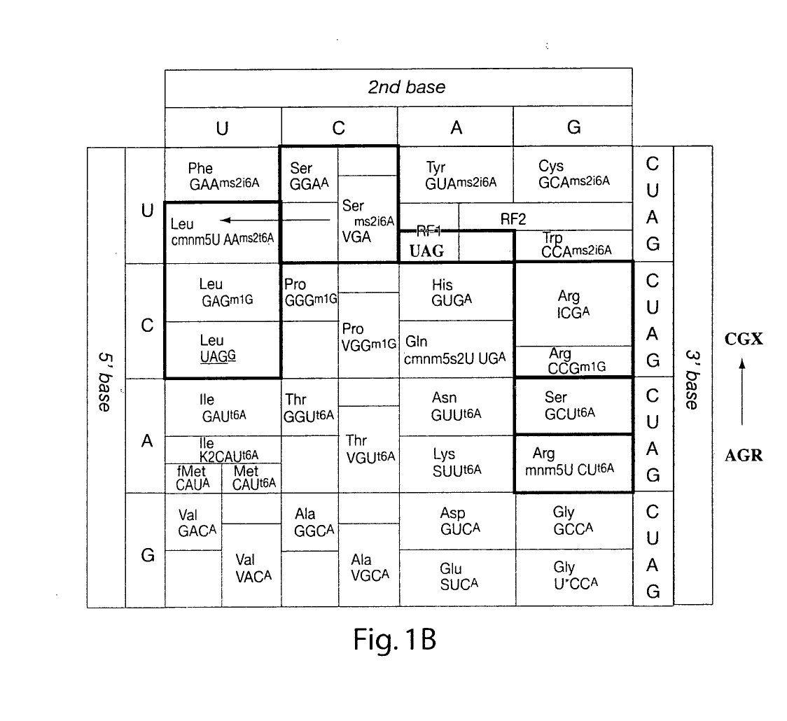

In certain embodiments, the predetermined codon is a codon recognized by a natural tRNA charged with one of the 20 canonical amino acids. In other embodiments, the predetermined codon is a terminator codon. However, it should be appreciated that aspects of the invention include templates where two or more predetermined codons are selected for incorporating two or more different unnatural amino acids. The selected codons may be coding codons, stop codons, or a combination thereof. If a selected codon is a coding codon, the host organism may be modified further to reduce or abolish the expression of one or more natural tRNA molecules that recognize that codon and may compete with the artificial tRNA for amino acid incorporation at that position. If a selected codon is a stop codon, the host organism may be modified further to reduce or abolish expression of one or more termination factors (e.g., release factors) that may compete with the modified tRNA to terminate translation at that position. It should be appreciated that several factors should be considered when selecting a predetermined codon that will be used to specify an unnatural amino acid in an artificial expression system of the invention. It may be preferable to chose a codon that is under-represented in the genomic open reading frames of the host organism in order to minimize the number of genomic changes that may be required to promote incorporation of the unnatural amino acid(s) into the polypeptide of interest. It may be preferable to chose a codon that is infrequent in the open reading frame of the template nucleic acid. It may be preferable to chose a codon that is not recognized by a plurality of different tRNAs in order to avoid natural tRNAs interfering of competing with the modified tRNA, or in order to avoid additional modifications to the host genome to remove or reduce the expression of the natural tRNAs that recognize the selected codon. It may be preferable to use a codon for which an appropriate modified tRNA is readily available (e.g., for which an appropriate modified tRNA synthetase is readily available). It may be preferable to use a stop codon to avoid competing with natural tRNAs. It may be preferable to use a codon that is under-represented in the host organism (e.g., a stop codon such as UAG in E. coli) in order to reduce the number of genomic changes that may be required to promote incorporation of the unnatural amino acid(s) into the polypeptide of interest. It may be preferably to use a weaker stop codon or use a host strain in which the strength of a particular stop codon is reduced due to one or more genetic alterations (e.g., mutations, deletions, etc.). It should be appreciated that the selection of a predetermined codon may require weighing these competing factors in order to determine the most appropriate codon. In some instances, two or more codons may be tested and compared in order to determine the most effective one to use. FIG. 1A shows the relative numbers of different codons (including stop codons) in E. coli. Codons that are relatively infrequent are good candidates for replacement. Examples include UUR (TTR) (R is A or G), UAG (TAG), AGR, AGY (Y is T, C, or U). However, any of the codons may be replaced if appropriate. In particular, any of the stop codons may be used. However, UAG may be preferred in certain embodiments since it is less frequent than either of the other two stop codon. FIG. 1B further shows an analysis of codons that are readily freed up in E. coli. Similar determinations may be made in other organisms.

In certain embodiments, reducing the number of predetermined codons in the non-template open reading frames may be achieved by replacing one or more of the predetermined codon(s) in one or more non-template open reading frames with one or more alternative codon(s) that are recognized by the modified tRNA with lower efficiency and/or specificity than the predetermined codon(s). In one embodiment, the alternative codon(s) are not recognized by the modified tRNA. In one embodiment, the selected codon is a codon that encodes a natural amino acid in a normal translation system. In this instance, the alternative codon(s) introduced to replace the selected codon in the non-template open reading frame(s) may be one of the different degenerate codons that encode the same natural amino acid as the original codon. However, a codon for a different amino acid may be used, especially if it does not have a negative effect on the translation system (e.g., it does not have a deleterious effect on the host cell that would reduce growth or expression of the polypeptide of interest). In one embodiment, the selected codon is a stop codon. In this instance, the alternative codon should be one of the other two stop codons. Also, the open reading frame of the template should be modified (if necessary) to use one of the alternative codons to terminate the open reading frame.

It should be appreciated that the number of predetermined codons that are replaced in the genome of the host may be dependent on how many are required in order to obtain the desired improvement in unnatural amino acid incorporation and also on the effect that the codon replacements have on the viability, growth, protein expression, and/or other functional characteristics of the host organism. It may be more important to replace certain codons than others. For example, it may be more helpful to replace the predetermined codon(s) in highly expressed open reading frames rather than in open reading frames that are expressed either at low levels or only under certain growth conditions (e.g., heat shock). However, different embodiments of the invention include host organisms having different subsets of predetermined codons replaced. In some embodiments, more than about 1%, 5%, 10%, 20%, 30%, 40%, 50%, 60%, 70%, 80%, 90% or up to 100% of one or more predetermined codons are replaced with alternative codons. The actual number of codons that are replaced may be different for any given codon since the codon usage varies as shown in FIG. 1 for E. coli.

It should be appreciated that an improvement in unnatural amino acid incorporation may result in an increased amount of expression of artificial polypeptide containing the unnatural amino acid, increased efficiency of incorporation (e.g., less unnatural amino acid is required to obtain the same amount of artificial polypeptide containing the unnatural amino acid), or a combination thereof.

These and other aspects of the invention are described in more detail herein.

Methods of Expressing Artificial Polypeptides in Host Cells:

Aspects of the invention provide methods and compositions for promoting the incorporation of one or more unnatural amino acids into one or more predetermined positions in a polypeptide. Methods of the invention may be used in connection with a translation system using a modified tRNA that is charged with an unnatural amino acid and that recognizes a predetermined codon. According to the invention, a predetermined polypeptide may be expressed containing an unnatural amino acid by using an open reading frame that contains the predetermined codon recognized by the modified tRNA.

In one aspect, an unnatural amino acid is incorporated into a polypeptide during synthesis in vivo in a host cell. A polypeptide may be expressed in the host cell in the presence of a modified tRNA that is charged with the unnatural amino acid and that recognizes at least one predetermined codon that is present in an open reading frame encoding the polypeptide. The modified tRNA preferably is recognized efficiently and/or specifically by the translational machinery (e.g., ribosome and associated molecules) so that the unnatural amino acid is efficiently and/or specifically incorporated into the polypeptide at the appropriate position.

In one aspect, the invention provides a method for expressing an artificial polypeptide using a host cell that has been engineered to remove or replace one or more codons in the host genome that may interfere with efficient, accurate, and/or high level expression of the artificial polypeptide. One or more genomic open reading frame codons that are the same as the predetermined codon chosen for unnatural amino acid incorporation are removed or replaced so that they do not compete for the modified tRNA charged with the unnatural amino acid. For example, the host organism may be engineered to replace one or more of the predetermined codons that are present in one or more highly expressed open reading frames on the genome of the host with one or more alternative codons that do not interfere with the growth and or protein expression properties of the host.

Accordingly, one aspect of the invention provides a method of expressing an artificial polypeptide comprising at least one unnatural amino acid by expressing the polypeptide in a host cell in the presence of a first modified tRNA charged with a first unnatural amino acid, wherein the host cell comprises a) a genome that is modified to replace at least a threshold number of copies of a first codon with one or more first alternative codons, wherein the first modified tRNA recognizes the first codon with greater specificity than any of the one or more first alternative codons; and b) a first nucleic acid comprising an open reading frame having at least one copy of the first codon, wherein translation of the open reading frame results in expression of an artificial polypeptide comprising at least one unnatural amino acid.

In another aspect, an artificial polypeptide comprising at least one unnatural amino acid may be obtained by isolating it from a host cell expression system described herein.

It should be appreciated that the host cell may be any suitable cell, for example a bacterial cell, a yeast cell, an insect cell, a mammalian cell, a plant cell, a fungal cell, or any other prokaryotic or eukaryotic cell that can be manipulated according to aspects of the invention. The host cell may be an immortalized cell. In certain embodiments, the host cell may be a bacterial cell such as an E. coli cell. The host cell may be grown in culture (e.g., in suspension, in a shaker, in a fermentor, on a growth surface, or using any other suitable format). The host cell also may be part of a multicellular organism and the artificial polypeptide may be expressed in all, or a subset, of the cells in a multicellular organism such as a mammal or a plant (e.g., a farm animal or agricultural plant).

The threshold number of codons on the genome that are replaced with alternative codons is preferably a number above which increased incorporation of the unnatural amino acid into the artificial polypeptide is observed. The threshold number may depend on the identity of the codon that is being replaced. For example, the number of UAG stop codons (326) on the genome of E. coli cells is lower than the number of UAA or UGA stop codons (2703 and 1256, respectively). Therefore, an expression system using the UAG codon as the predetermined codon for incorporating unnatural amino acids into a polypeptide may require fewer genomic changes to remove interfering stop codons (UAG codons in this embodiment) from the genome than may be required by a counterpart system that uses a UAA or UGA stop codon as the predetermined codon. The threshold number also may depend on the identity of the open reading frames in which the codons are being replaced. In certain embodiments, codons may be replaced in one or more open reading frames encoding essential proteins and/or one or more open reading frames that are highly expressed. In some embodiments, codons may be replaced in one or more ribosomal protein genes. Certain open reading frames are more sensitive to codon replacement than others. In certain embodiments, one or more (e.g., about 5, 10, 25, 50, 100, 250, 500, 1,000, or more) copies of the first codon may be replaced. In certain embodiments, at least half of the first codons on the genome are replaced with one or more alternative codons. In certain embodiments, a substantial portion of the first codons on the genome are replaced with one or more alternative codons. In some embodiments, all of the first codons are replaced with one or more alternative codons.

It should be appreciated that methods of the invention may be used to incorporate two or more copies of an unnatural amino acid into a polypeptide by including two or more copies of the predetermined codon in the target open reading frame. It also should be appreciated that methods of the invention may be used to incorporate two or more different unnatural amino acids into a polypeptide by including two or more different predetermined codons in the target open reading frame, wherein each different predetermined codon is recognized by a different modified tRNA charged with a different unnatural amino acid. Similarly, three or more different unnatural amino acids may be incorporated into an artificial polypeptide using three or more different predetermined codons. One or more copies of each predetermined codon (e.g., 2, 3, 4, 5, 10, or more) may be included as appropriate as the invention is not limited in this respect.

It should be appreciated that when two or more different predetermined codons are used to incorporate two or more different unnatural amino acids into a polypeptide, the host organism may be modified to replace at least one copy of at least one of the predetermined codons in the genome of the host. In certain embodiments, at least one copy of each of the predetermined codons is removed from an expressed open reading frame on the genome of the host. The threshold number of each codon that should be replaced may be determined by the number above which increased expression of the unnatural polypeptide is obtained. The threshold number for each codon may be different.

It should be appreciated that a predetermined codon may be chosen based on the number of copies of that codon that are present in the host genome. For example, it may be preferable to chose a codon that is not heavily used by the host organism so that not many changes are needed to the genome of the host.

In certain embodiments, the predetermined first codon may be one of several degenerate codons for a natural amino acid. In other embodiments, the predetermined first codon may be a first stop codon that is recognized by the first modified tRNA, and the open reading frame is terminated by a stop codon that is different from the first stop codon. The first stop codon may be UAA, UAG, or UGA. However, it should be appreciated that the open reading frame may comprise at least one copy of a second stop codon that is recognized by a second modified tRNA in the host cell, and wherein the open reading frame is terminated by a third stop codon that is different from the first and second stop codons.

In one embodiment, the first predetermined codon is UAA, the open reading frame is terminated by an alternative stop codon, and the genome of the host cell comprises at least a threshold number of UAA codons replaced with an alternative stop codon. The alternative stop codon may be UAG or UGA. In one embodiment, a fraction of the threshold number of UAA codons may be replaced by UAG and the remainder of the threshold number of UAA codons may be replaced with UGA.

In one embodiment, the first predetermined codon is UAG, the open reading frame is terminated by an alternative stop codon, and the genome of the host cell comprises at least a threshold number of UAG codons replaced with an alternative stop codon. The alternative stop codon may be UAA or UGA. In one embodiment, a fraction of the threshold number of UAG codons may be replaced with UAA and the remainder of the threshold number of UAG codons may be replaced with UGA.

In one embodiment, the first predetermined codon is UGA, the open reading frame is terminated by an alternative stop codon, and the genome of the host cell comprises at least a threshold number of UGA codons replaced with an alternative stop codon. The alternative stop codon may be UAG or UAA. In one embodiment, a fraction of the threshold number of UGA codons may be replaced with UAG and the remainder of the threshold number of UGA codons may be replaced with UAA.

In one embodiment, the first predetermined codon is UAA, the second predetermined codon is UGA, and the open reading frame is terminated by UAG. The genome of the host cell may be modified to comprise at least a threshold number of UAA codons replaced with UAG. The genome of the host cell also may be modified to comprise at least a threshold number of UGA codons replaced with UAG. Accordingly, the genome of the host cell may be modified to comprise at least a first threshold number of UAA codons replaced with UAG and at least a second threshold number of UGA codons replaced with UAG.

In one embodiment, the first predetermined codon is UAA, the second predetermined codon is UAG, and the open reading frame is terminated by UGA. The genome of the host cell may be modified to comprise at least a threshold number of UAA codons replaced with UGA. The genome of the host cell also may be modified to comprise at least a threshold number of UAG codons replaced with UGA. Accordingly, the genome of the host cell may be modified to comprise at least a first threshold number of UAA codons replaced with UGA and at least a second threshold number of UAG codons replaced with UGA.

In one embodiment, the first predetermined codon is UAG, the second predetermined codon is UGA, and the open reading frame is terminated by UAA. The genome of the host cell may be modified to comprise at least a threshold number of UAG codons replaced with UAA. The genome of the host cell also may be modified to comprise at least a threshold number of UGA codons replaced with UAA. Accordingly, the genome of the host cell may be modified to comprise at least a first threshold number of UAG codons replaced with UAA and at least a second threshold number of UGA codons replaced with UAA.

It should be appreciated that if the first codon is UAA or UAG and the host is a prokaryote (e.g., E. coli), it may be helpful to use a modified genome comprising a mutation that reduces expression of release factor 1. Similarly, if the first codon is UAA or UGA, and the host is a prokaryote (e.g., E. coli), it may be helpful to use a modified genome comprising a mutation that reduces expression of release factor 2. Equivalent translational mutations may be introduced into other host organisms in order to further promote incorporation of unnatural amino acids at predetermined locations in artificial polypeptides expressed from template nucleic acids.

In embodiments of the invention, the replacement of a plurality of predetermined codons in portions of the host genome, or throughout the host genome, may be accomplished using one or more methods described herein, including large scale synthesis of one or more synthetic genome regions, incorporation and recombination of large nucleic acid regions into the genome of the host organism, and/or sequential or hierarchical nucleic acid replacement strategies described herein.

Polypeptide Isolation:

Aspects of the invention relate to methods for isolating polypeptides containing one or more unnatural amino acids. A polypeptide may be isolated from a preparation that was generated using an expression system (e.g., an in vivo expression system) described herein. In certain embodiments, the invention provides different forms of preparations containing an artificial polypeptide with at least one unnatural amino acid. Useful preparations for isolating an artificial polypeptide may include one or more of the following: a crude cell lysate, a cell pellet, a cell culture supernatant, a cell suspension, or any combination thereof which contains an artificial polypeptide expressed according a method described herein. It should be appreciated that these preparations may include relatively high amounts of artificial polypeptide since the invention provides methods for improved expression of these artificial polypeptides. In one embodiment, a crude cell lysate comprises an artificial protein containing at least one unnatural amino acid, wherein the artificial protein represents at least a threshold percentage of total protein material in the crude cell lysate. The artificial protein may represent at least a threshold percentage of total protein material that contains the at least one unnatural amino acid in the crude cell lysate. In one embodiment, the artificial protein may be the only protein that contains the unnatural amino acid in the crude cell lysate. Similarly, a cell culture supernatant may comprise an artificial protein containing at least one unnatural amino acid, wherein the artificial protein represents at least a threshold percentage of total protein material in the cell culture supernatant. The artificial protein may represent at least a threshold percentage of total protein material that contains the at least one unnatural amino acid in the cell culture supernatant. In one embodiment, the artificial protein may be substantially the only protein that contains the unnatural amino acid in the cell culture supernatant.

In the embodiments described above, the threshold amount may be about 0.1%, about 1%, about 5%, about 10%, about 25%, about 50%, about 75%, or about 100%. However, intermediate threshold amounts also are contemplated.

Polypeptide Compositions:

Certain aspects of the invention relate to compositions comprising artificial polypeptides containing one or more unnatural amino acids. In certain embodiments, an unnatural amino acid includes an unnatural side chain with a bioorthogonal functional group that does not interfere with biological reactions and does not react significantly, if at all, with biological molecules. However, a bioorthogonal functional group may react with other bioorthogonal functional groups under appropriate conditions to generate covalent bonds via pericyclic reactions (e.g., cycloaddition), condensation reactions, nucleophilic additions, and other reactions. In some embodiments, other types of unnatural amino acid side chains may be incorporated into a polypeptide. For example, one or more biologically reactive functional groups may be used when a polypeptide is designed to interact with other biological molecules (e.g., other proteins, lipids, carbohydrates, etc.). Alternatively, an essentially inert unnatural amino acid may be used when certain structural features may be required (e.g., to stabilize a polypeptide structure or to relocate or rearrange a polypeptide structural element) without introducing any functional reactivity (with either biological or non-biological molecules) under biological, purification, or storage conditions.

The introduction of bioorthogonal functional groups into an artificial polypeptide may be used to form non-native intramolecular bonds within the polypeptide (e.g., involving reactions between two or more bioorthogonal functional groups) or non-native intermolecular bonds between other molecules (e.g., other molecules containing one or more bioorthogonal functional groups) and one or more bioorthogonal functional groups within an artificial polypeptide.

In certain embodiments, an artificial polypeptide may include a covalent bond between a side chain of a first unnatural amino acid and a side chain of a second amino acid in the artificial polypeptide. The second amino acid also may be an unnatural amino acid. In further embodiments, the artificial polypeptide may include a covalent bond between a side chain of a first unnatural amino acid and the N-terminal end of the artificial polypeptide. In yet further embodiments, the artificial polypeptide may comprises a covalent bond between a side chain of a first unnatural amino acid and the C-terminal end of the artificial polypeptide. The covalent bond in any of these polypeptides may be internal to a folded tertiary structure of the polypeptide. The covalent bond may be formed intra-cellularly. Alternatively, the covalent bond may be formed extra-cellularly (e.g., by exposing the isolated artificial polypeptide to air, humidity, and/or a catalyst that promotes covalent bond formation (e.g., a metal ion such as Cu(I) or other suitable catalyst.

In some embodiments, an internal covalent bond in an artificial polypeptide is formed by the reaction of one or more (e.g., 2, 3, 4, 5, or more) unnatural amino acids having functional groups that are substantially bioorthogonal (groups that are stable and inert to functional groups which are typically found in biological systems). In some embodiments, two bioorthogonal groups react selectively with each other to form a non-native covalent bond (i.e., a bond between two unnatural functional groups, unlike a Cys-Cys disulfide bond). In one embodiment, the reactive groups and the product formed from reaction between the reactive groups do not disturb the three-dimensional structure of, for example, the artificial protein in which they are incorporated.

A reaction for generating an internal non-native covalent bond may occur when two bioorthogonal reactive groups are positioned in relatively close proximity to one another (e.g., they are located at positions on the polypeptide chain that are in relatively close proximity in the folded polypeptide structure). In some cases, the reaction may have a thermodynamic driving force of at least 20 kcal/mol and may by performed in relatively mild conditions (e.g., at relatively neutral pH, in biocompatible solvents, at room temperature, etc.). Also, the reaction may produce byproducts which are nontoxic and inert to the other functional groups found in biological systems. Alternatively, the reaction may produce no byproducts. Accordingly, a bioorthogonal reaction may occur intracellularly. However, a bioorthogonal reaction may occur or be promoted extracellularly (e.g., after purification) by exposing an artificial polypeptide to one or more specific catalysts or to conditions (e.g., appropriate pH, salt, temperature, etc.) that favor the reaction. Certain useful bioorthogonal reactions, including click chemistry, are described in the art. See, for example, Prescher and Bertozzi, Chemistry in living systems, (2005) Nature Chemical Biology (1) 1:13-21, or Kolb and Sharpless, The growing impact of click chemistry on drug discovery, (2003) Drug Discovery Today (8)24:1128-1137. However, other appropriate chemistries and unnatural functional groups (functional groups not found naturally in biological systems) may be used as described herein.

In one embodiment, two bioorthogonal groups may perform a cycloaddition. The term “cycloaddition” is known the art and refers to a pericyclic reaction in which a new covalent bond is formed. The product formed by the cycloaddition may be a ring structure of any size, such as three-membered, four-membered, five-membered, six-membered, and the like. For example, two reactive groups may perform a 1,3-dipolar cycloaddition to form a five-membered ring. In some cases, a metal ion (e.g., a copper ion) may be used to facilitate the cycloaddition. The cycloaddition reaction may involve reaction of a carbon-carbon double bond (e.g., an alkene) or a carbon-carbon triple bond (e.g., an alkyne) with a 1,3 dipolar compound. Examples of 1,3-dipolar compounds include azide, diazoalkanes, nitrous oxides, nitrile imines, nitrile ylides, nitrile oxides, azomethine imines, azoxy compounds, azo methine ylides, nitrones, and the like. In one embodiment, an azide and a carbon-carbon triple bond (e.g., an acetylene group) may react to form a triazole ring. Examples of other cycloadditions include Diels-Alder reactions, (e.g., [4+2]cycloadditions), hetero-Diels-Alder reactions, ene reactions, and the like.

In some embodiments, a highly strained ring may perform a cycloaddition with an alkene or alkyne to form a less strained ring. For example, a cyclopropene ring may perform a cycloaddition with an alkene to form a cyclopentene ring, where the driving force of the reaction is the release of ring strain.

In some embodiments, two bioorthogonal groups may perform a condensation reaction. As used herein, a “condensation reaction” refers to a chemical reaction in which at least two moieties react and become covalently bonded to one another resulting in the loss of a small molecule, such as water. In some embodiments, one of the moieties is a carbonyl compound, such as an aldehyde, a ketone, a carboxylic acid, or an ester. Examples of condensation reactions include the reaction of an alehyde or a ketone with an amine or substituted amine to form an imine or enamine, respectively, the reaction between an azide and an ester to form an amide, the reaction between a ketone or aldehyde and a hydrazine, substituted hydrazine, or hydrazide to form a hydrazone, the reaction between a keton or aldehyde and an aminooxy group to form an oxime or oxime ether, the like. Although aldehydes and ketones may reversibly react with primary amines present in natural amino acids (e.g., lysine), the equilibrium favors the carbonyl. In contrast, the equilibrium favors hydrazones and oxime ethers. Other reactions involving carbonyl compounds may also form aromatic heterocycles and ureas.

In other embodiments, the reactive groups may perform a nucleophilic ring opening of a strained ring (e.g., three-membered rings). Examples of strained rings include epoxides, aziridines, and episulfides, and substituted derivatives thereof. The driving force in the nucleophilic addition to a carbon of such highly strained rings may be the release of ring strain. Such reactions may proceed with regioselectivity and stereoselectivity. Examples of nucleophilic groups which may add to a carbon of such strained rings include

In some embodiments, the side chain of an unnatural amino acid may form an intermolecular covalent bond to an external molecule. The side chain may comprise a functional group which may be modified by a selective reaction with a target molecule, such as a molecular probe, containing a complementary functional group. The functional group of the side chain may be inert to reactive groups typically found in proteins and other biomolecules. Also, the functional group of the side chain may be relatively small to avoid structural perturbation of the biomolecule (e.g., protein) to which it is bonded. Examples of such functional groups include ketones, aldehydes, alkynes, alkenes, azides, hydrazides, aminooxy groups, and the like.

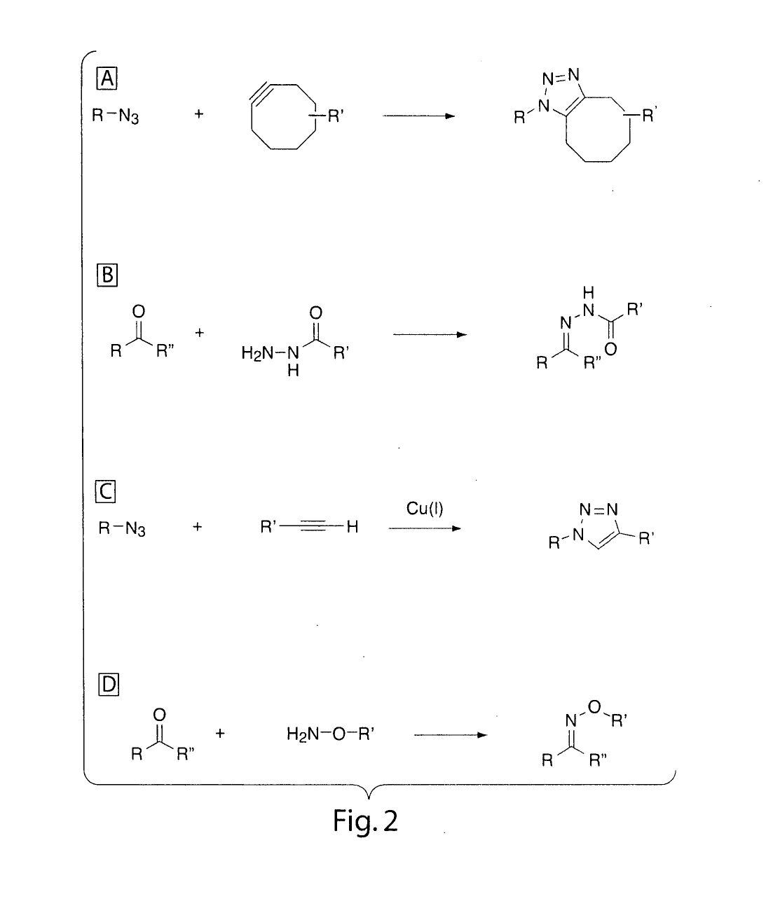

Non-limiting examples of reactions that may be used to form non-native intramolecular and/or intermolecular covalent bonds are shown in FIG. 2.

When selecting the appropriate functional group(s) to include in one or more unnatural amino acids (e.g., for selective intramolecular or intermolecular bond formation) the unnatural functional group may be selected, at least in part, based on the size of the functional group, the size or length of a linker connecting the functional group to the alpha carbon of the unnatural amino acid, the position at which the unnatural amino acid is incorporated in the artificial polypeptide chain, the size and chemical properties of the natural amino acid that is being replaced. For example, if a hydrophobic amino acid is being replaced at a position that is internal in a folded polypeptide chain, an unnatural amino acid with similar size and hydrophobic properties may be selected. However, if the unnatural amino acid is going to form an internal covalent bond with another amino acid, the chemical properties of the unnatural amino acid may be different from the natural one if, for example, the strength of the non-native covalent bond compensates for the loss of hydrophobicity. Regardless of changes in properties such as hydrophobicity, polarity, electric charge, etc., the size of the unnatural amino acid should be compatible with the folded structure of the polypeptide. Certain proteins (or polypeptide functional or structural domains) are highly sensitive to minor structural changes, and only unnatural amino acids with essentially the same structural properties as the natural amino acids being replaced may be used. Other proteins (or polypeptide functional or structural domains) are less sensitive to minor structural changes, and unnatural amino acids with a range of sizes may be used to replace certain natural amino acids. When replacing a buried amino acid, it may be more important to use an unnatural amino acid of essentially the same size that when replacing a surface or surface exposed amino acid. However, information about the protein structure (e.g., from crystallography, NMR, or structure software analysis) may be used to determine the appropriate unnatural amino acid to select (or a range of appropriate amino acids to test in order to find one with the desired functional and/or structural effect on a polypeptide of interest). In some embodiments, an buried salt bridge may be replaced by a non-native covalent bond between bioorthogonal amino acids. Similarly, non-native covalent bonds may be introduced to replace one or more Cys-Cys disulfide bridges. However, non-native covalent bonds may be introduced at positions where no significant structural bonds were present in a natural protein (other than hydrophobic or packing interactions) in order to stabilize or otherwise alter the structure or function of the protein.

Accordingly, aspects of the invention may be used for incorporating any appropriate unnatural amino acid into a polypeptide of interest to produce an artificial polypeptide with altered functional and/or structural properties. As discussed, an unnatural amino acid may be an amino acid that contains a bioorthogonal unnatural side chain functional group, a bioreactive unnatural side chain functional group, or even an inert unnatural side chain functional group. The side chain may a substituted natural amino acid side chain. Examples of unnatural amino acid side chains and unnatural amino acids include, but are not limited to one or more of the following.

Examples of unnatural amino acids include O-methyl-L-tyrosine; L-3-(2-naphthyl)alanine; 3-methyl-phenylalanine; 3-nitro-L-tyrosine; O-4-allyl-L-tyrosine; 4-propyl-L-tyrosine; O-(2-propynyl)-L-tyrosine; tri-O-acetyl-GlcNAcβ-serine; L-Dopa; fluorinated phenylalanine; isopropyl-L-phenylalanine; p-azido-L-phenylalanine; p-acyl-L-phenylalanine; p-benzoyl-L-phenylalanine; L-phosphoserine; phosphonoserine; phosphonotyrosine; p-iodo-phenylalanine; p-bromophenylalanine; p-amino-L-phenylalanine; p-nitro-L-phenylalanine; p-isopropyl-L-phenylalanine; m-methoxy-L-phenylalanine; p-acetyl-L-phenylalanine; m-acetyl-L-phenylalanine; p-ethylthiocarbonyl-L-phenylalanine; p-(3-oxobutanoyl)-L-phenylalanine; dihydroxy-L-phenylalanine; para-azidophenylalanine; p-carboxy-methyl-L-phenylalanine; azidohomoalanine; homoglutamine; 2-aminooctanoic acid; beta-N-acetylglucosamine-O-serine; and alpha-N-acetylgalactosamine-O-threonine.

Examples of side chain functional groups include alkyl; aryl; acyl; azido; cyano; halo; hydrazine; hydrazide; hydroxyl; alkenyl; alkyl; ether; thiol; sulfonyl; seleno; ester; thioacid; borate; boronate; phospho; phosphono; phosphine; heterocyclic; enone; imine; aldehyde; hydroxylamine; and keto groups.

Examples of unnatural amino acids based on natural amino acids include: tyrosine-analogs: para-substituted tyrosines; ortho-substituted tyrosines; meta-substituted tyrosines, wherein the substituted tyrosine comprises an acyl group; benzoyl group; amino group; hydrazine; hydroxylamine; thiol group; carboxyl group; isopropyl group; methyl group; C₆-C₂₀ straight chain or branched hydrocarbon; saturated or unsaturated hydrocarbon; O-methyl group; polyether group; nitro group; and multiply substituted aryl rings; glutamine analogs: α-hydroxyl derivatives, β-substituted derivatives, cyclic derivatives, amide substituted derivatives; phenylalanine analogs: meta-substituted phenylalanines, wherein the substituent comprises a hydroxy group; methoxy group; methyl group; alkyl group; acetyl group; and other suitable amino acid analogs.

It should be appreciated that different unnatural amino acids may be incorporated into different artificial polypeptides for different purposes. One or more unnatural side chains may impart one or more beneficial properties on an artificial polypeptide. In one embodiment, one or more unnatural side chains may form covalent bonds that stabilize the artificial polypeptide (e.g., stabilize a functionally or structurally active form of the polypeptide). Certain unnatural amino acids have side chains that may be useful for forming internal covalent bonds that may stabilize a folded protein. In contrast, certain unnatural amino acids have side chains that may be useful for adding one or more reactive functions to the surface of a protein. For example, certain unnatural side chains, if exposed on the surface of a protein, may render the protein more susceptible to one or more of the following chemical modifications: acetylation; amidation; biotinylation; dabcylation; dansylation; dinitrophenylalanine; amino (DNP); methylation (mono-, di-, tri-); myristoylation; palmitoylation; prenylation (farnesylation, geranylgeranylation); phosphorylation; succinylation; sulfonation; formylation; benzyloxycabonylation; or pegylation.

In certain embodiments, one or more unnatural reactive groups may be used for modifying a protein (e.g., after isolation or purification) in order to improve its therapeutic properties. The therapeutic properties may be improved simply by improving the solubility and or stability of the therapeutic protein thereby improving its bioavailability. However, the therapeutic properties also may be improved by altering certain functional or structural features of the polypeptide that may alter certain specific interaction with other biological molecules. It should be appreciated that the type of unnatural amino acid and the position at which it is added will be determined based on several factors including, but not limited to, the structure and composition of the protein (and whether it contains more than one different unnatural amino acid) and/or the intended purpose of the modification (e.g., to stabilize, to increase reactivity, etc.). In some embodiments, an unnatural amino acid may help target a protein (e.g., a therapeutic protein) to a site of action (e.g., to a particular tissue or organ in a patient). For example, the unnatural amino acid side chain may itself target an artificial polypeptide to an organ or tissue (e.g., CNS, liver, gut, etc.). Alternatively, the unnatural amino acid may provide a suitable reactive group that can be modified by a addition of a moiety (e.g., a carbohydrate, lipid, or other suitable moiety) that targets the artificial polypeptide to an organ or tissue.

In some embodiments, the artificial polypeptide may be a modified form of a known natural or recombinant protein (e.g., a therapeutic protein or a biologically active protein). The unnatural amino acid may be incorporated by replacing a natural amino acid in the polypeptide chain. Alternatively, the unnatural amino acid may be added to the polypeptide chain without replacing any natural amino acids. It should be appreciated that structural and functional information about proteins and unnatural amino acids may be used to determine one or more candidate modifications for any intended purpose. However, in some instances several candidate artificial proteins with one or more different unnatural amino acids may need to be produced and assayed in order to identify a suitable modification for a chosen application. Using methods of the invention, several different artificial proteins may be readily expressed and isolated for one or more functional assays.

Accordingly, some embodiments of the invention provide an artificial protein comprising an intramolecular covalent bond between a side chain of a first unnatural amino acid and a side chain of a second amino acid, wherein the covalent bond is an internal bond in a folded form of the artificial protein. The second amino acid may be unnatural. The first and second unnatural amino acids may be different. Either one or both of the first and second amino acids may be buried in a folded form of the artificial protein. However, a portion of either one or both of the first and second amino acids may be solvent exposed in a folded form of the protein (e.g., partially buried by 10-30%, 30-50%, 50-70%, or 70-90%). In certain embodiments, an artificial protein may include two or more covalently bonded amino acid pairs, wherein at least one amino acid in each covalently bonded amino acid pair is unnatural. In some embodiments, a covalent bond may thermodynamically stabilize a folded structure of an artificial protein relative to a corresponding folded structure of a natural or recombinant protein that contains only natural amino acids. The folded structure may be a biologically active form of the artificial protein. The covalent bond may stabilize a first form relative to a second form of an allosteric protein. In certain embodiments, an artificial protein comprises an internal covalent bond that stabilizes a secondary structural motif (e.g., an alpha helix or a beta sheet). In certain embodiments, an artificial protein comprises an internal covalent bond that stabilizes a tertiary structural fold of the artificial protein.

In some embodiments, an artificial protein may have an increased half-life relative to a corresponding natural or recombinant protein that contains only natural amino acids. The increased half-life may be an increased shelf life in a storage buffer. The increased half-life may be an increased half-life in a pharmaceutical preparation. The increased half-life may be an increased in-vivo half-life of the artificial protein after administration to a patient. The increased half-life may be an increased circulating half-life in a human.

In certain embodiments, an artificial polypeptide may be isolated from an expression system using one or more of the methods described herein. The resulting preparation may contain relatively large amounts of artificial polypeptide. For example, methods of the invention may be used to make large scale or industrial amounts of artificial polypeptides containing one or more unnatural amino acids. Large scale amounts may include about 10 g, 100 g, 1 kg, 10 kg, 100 kg, or more of the artificial polypeptide.

The polypeptide may be purified to more than 50%, 60%, 70%, 80%, 90%, 95%, 99% or greater purity.

An artificial polypeptide of the invention may be of any desired length. For example, the artificial polypeptide may be at least 50 amino acids long. The artificial polypeptide may consist essentially of between 100 and 200 amino acids, between 200 and 500 amino acids, or between 500 and 1,000 amino acids. The artificial polypeptide may be more than 1,000 amino acids long.

An artificial polypeptide of the invention may be provided in an essentially pure dry or powdered form (e.g., lyophilized). An artificial polypeptide of the invention also may be provided in a composition such as a solution, a solid, a gel, a powder, a colloid, a precipitate, etc. that also may include one or more additional compounds. In one embodiment, the composition comprises a buffer, for example a storage buffer or a physiologically acceptable buffer. The composition may include a therapeutically effective amount of the artificial polypeptide. However, the composition may include an amount that is above the therapeutically effective amount and may need to be diluted prior to use (e.g., in therapy). The composition may be sterilized.

In certain embodiments, an artificial polypeptide of the invention may be provided in a pharmaceutical preparation comprising a pharmaceutically acceptable excipient, diluent, or carrier (e.g., a polymer). The pharmaceutical preparation also may include a therapeutic agent including, but not limited to, one or more of the following: an antibiotic, a chemotherapeutic, an immunomodulatory, or other therapeutic agent.

An artificial protein may be a modified form of a known therapeutic or biologically active natural or recombinant protein (e.g., a hormone, an antibody, an enzyme, a receptor, an antigen, a vaccine, an antibiotic, a small peptide, an interferon, an interleukin, etc.). For example, an artificial protein may be a modified form of a natural or recombinant therapeutic protein such as insulin, EPO, growth hormone (e.g., hGH). Other examples of known proteins or peptides that may be modified and expressed according to methods of the invention include, but are not limited to: interleukins, including IL-1-like interleukins such as IL-1α:, IL-1β, IL-IRA, IL-18; common g chain (CD132) molecules such as IL-2, IL-4, IL-7, IL-9, IL-13, IL-15, common b chain (CD131) molecules such as IL-3, IL-5, other interleukin-like molecules including GM-CSF, IL-6-like, IL-6, IL-11, G-CSF, IL-12; LIF, OSM, IL-10-like, IL-10, IL-20, IL-14, IL-16, IL-17; interferon such as IFN-α, IFN-β, IFN-γ; other molecules including TNF; CD154; LT-β; TNF-α; TNF-β; 4-1BBL; APRIL; CD70; CD153; CD178; GITRL; LIGHT; OX40L; TALL-1; TRAIL; TWEAK; TRANCE; TGF-β like molecules including TGF-β1, TGF-β2, TGF-β3; hematopoietins such as EPO, TPO, Flt-3L, SCF, M-CSF, MSP; chemokines including C chemokines such as lymphoactin a, SCM-1a, ATAC, lymphoactin b, SCM-1b; CC chemokines such as I-309, MCP-1, MCAF, MIP-1α, LD78α, MIP-1β, LAG-1, ACT-2, RANTES, MCP-3, MCP-2, eotaxin, MCP-4, HCC-1, HCC-2, Lkn-1, MIP-Id, MIP-5, HCC-4, LEC, LMC, LCC-1, TARC, DC-CK1, PARC, AMAC-a, MIP-4, MIP-3β, ELC, exodus-3, MIP-3α, LARC, exodus-1, 6 Ckine, SLC, exodus-2, MDC, STCP-1, MPIF-1, MIP-3, CKb-8 MPIF-2, eotaxin-2, CKb-6, TECK, MIP-4-a, eotaxin-3, Eskine, CTACK, ILC; CXC chemokines such as ELR, GROa, MGSA-a, GROb, MGSA-b, MIP-2a, GROg, MGSA-g, MIP-2b, PF4, oncostatin A, ENA-78, GCP-2, NAP-2, PPBP, IL-8, NAP-1, NAF, MDNCF, Mig, IP-10, I-TAC, SDF-1α/β, BLC, BCA-1, BRAK, CX3C chemokines such as fractalkine; other growth factors; immunostimulatory molecules, cell cycle regulatory molecules, TK inhibitors; cetuximab (IMC-C225, Erbitux™), a monoclonal EGFR antibody; bevacizumab (Avastin™), a monoclonal antibody against VEGF; VEGFR2 inhibitor PTK787/ZK 222584; imatinib mesilate (STI-571, Gleevec™), an inhibitor of bcr-abl TK; an inhibitor of c-kit receptor TK; VEGFR2 inhibitor PTK787/ZK 222584; thalidomide; farnesyl transferase inhibitor R115777 (tipifarnib, Zarnestra™); matrix metalloproteinase inhibitors; bortezomib (Velcade™), a proteasome inhibitor; mammalian target of rapamycin (mTOR) inhibitors; cyclooxygenase-2 (COX-2) inhibitors; platelet derived growth factor receptor (PDGF-R) inhibitors; protein kinase C(PKC) inhibitors; mitogen-activated protein kinase (MEK) ½ inhibitors; Rous sarcoma virus transforming oncogene (SRC) kinase inhibitors; histondeacetylase (HDAC) inhibitors; small hypoxia-inducible factor (HIF) inhibitors; aurora kinase inhibitors, hedgehog inhibitors; TGF-beta signalling inhibitors; a chimeric monoclonal antibody against TNF-alpha; infliximab (Remicade) and etanercept, selective blockers of the cytokine tumor necrosis factor (TNF)-alpha; alefacept (Amevive) and efalizumab (Raptiva), T-cell modulators; etanercept (Enbrel), a soluble TNF receptor; anakinra, a recombinant human interleukin-1 receptor antagonist (IL-Ira); bevacizumab, an antivascular endothelial growth factor monoclonal antibody; adalimumab: TNF inhibitor; Platelet GP IIb/IIa receptor antagonists (GPAs): the human-murine chimeric monoclonal antibody Fab fragment abciximab, the peptide antagonist eptifibatide, the peptidomimetics tirofiban and lamifiban; chimeric monoclonal anti-TNF antibody (infliximab); a humanized monoclonal anti-TNF antibody (CDP571); a recombinant TNF receptor fusion protein (etanercept); inhibitors of TNFalpha activity; enfuvirtide (Fuzeon), a 36 amino acid peptide derived from the natural gp41 HR2 sequence (a HIV fusion inhibitor drug); T-1249, a 39 amino acid fusion inhibitor; anti-VEGF-antibody; fusion inhibitor T-20 or enfuvirtide; Celecoxib, a selective cyclooxygenase-2 (COX-2) inhibitor; atazanavir (Reyataz), amprenavir, indinavir, Kaletra (ritonavir+lopinavir), nelfinavir, ritonavir, and saquinavir-protease inhibitors; Ziconotide, a synthetic cone snail peptide varpi-conotoxin MVIIA: a neurone-specific N-type calcium channel blocker; bosentan (ETA/ETB antagonist); fully synthetic statin cerivastatin; inhibitors of angiotensin-converting enzyme (ACE) and neutral endopeptidase (NEP); imipramine; secretase inhibitors; inhibitors of acetylcholinesterase (AChE); mitoxantrone; Daptomycin, a cyclic lipopeptide; Cubicin (daptomycin for injection); quinupristin/dalfopristin; beta(2)-adrenergic agonists; calcium channel blockers; oxytocin antagonists; Zenapax®, a humanized monoclonal antibody against IL-2; Synagis®, a monoclonal antibody against RSV; AcuTect®, a synthetic peptide radiopharmaceutical; Thyrogen®, a recombinant human TSH; NeoTect®, a synthetic peptide radiopharmaceutical; Zevalin®, a radioimmunotherapeutic agent; palifermin (Kepivance®), a human keratinocyte growth factor; rituximab, a chimeric anti-human CD20 monoclonal antibody; Tysabri® (Natalizumab, formerly known as Antegren), a monoclonal antibody against alpha-4-integrin; and secretin.

Aspects of the invention may be used to express and produce artificial forms of any known polypeptide or protein, including invertebrate, vertebrate, insect, bacterial, plant, or any other polypeptide or protein. For example, any mammalian (including, human, mouse, horse, cow, pig, sheep, goat, etc.) protein may be modified and expressed as described herein.

However, aspects of the invention also may be used to express and produce designed polypeptides that are not derived from known polypeptides or proteins. It should be appreciated that designed polypeptides may include motifs from known proteins. In particular, any designed polypeptide may include a natural signal peptide in order to promote export or secretion of the polypeptide into a culture supernatant. Similarly, any artificial polypeptide of the invention (including those that are based on known natural or recombinant proteins) may be engineered to include an appropriate signal sequence for secretion in addition to the incorporation of one or more unnatural amino acids. In addition, a signal sequence may be modified according to methods of the invention (for example to stabilize it via the introduction of a non-native covalent bond, or to increase its hydrophobic properties vie the introduction of an unnatural amino acid with a very hydrophobic side chain.

Host Cells and Nucleic Acids:

Aspects of the invention also relate to host cells and nucleic acids that incorporate one or more features of the invention as discussed herein.

In certain embodiments, a host cell has been modified to have at least a threshold number of one or more genomic stop codons replaced by an alternative stop codon. As described herein, such host cells may further include additional modifications and/or nucleic acids to promote efficient incorporation of one or more unnatural amino acids into an artificial polypeptide expressed in the host cell.

In certain embodiments, the invention provides one or more nucleic acids (e.g., template nucleic acids) that are useful for producing artificial polypeptides containing one or more unnatural amino acids.

Applications:

Aspects of the invention may be useful for a range of different applications where proteins with altered functional or physical properties are desired.

As described herein, the invention provides methods for producing large amounts of artificial polypeptides containing unnatural amino acids, wherein the polypeptides were designed to have certain structural and or functional properties. As discussed, knowledge of three-dimensional structures and/or active site or other functional residues of certain proteins may be used to design or to guide the selection of certain amino acid changes that may stabilize, activate, inactivate or otherwise modify certain protein structures or functions.

In addition, aspects of the invention may be used to screen different amino acid changes and assay their effects on certain protein structural or functional properties. In one embodiment, a library of different host cells may be provided, each of which is designed to efficiently incorporate one or more different unnatural amino acid at one or more different codons. Accordingly, a single open reading frame could be expressed in different host cells to generate different artificial polypeptides that may be isolated and assayed. Alternatively, or in addition, an open reading frame for a selected protein may be modified in different ways to introduce different unnatural amino acids at different positions in the polypeptide chain. Again, the different artificial polypeptides may be isolated and assayed.

In another aspect, the invention provides a method of stabilizing a protein by introducing a stabilized secondary structural motif into the protein, wherein the stabilized secondary structural motif comprises a first unnatural amino acid with a side chain that is covalently bound the side chain of a second amino acid in the secondary structural motif.

In another aspect, the invention provides a method of making an expression construct for expressing a stabilized protein by introducing a nucleic acid cassette described herein into an open reading frame encoding a natural or recombinant protein. In some embodiments, a cassette encoding an artificial secondary structure containing an unnatural amino acid may be used to replace a sequence encoding a secondary structural motif in the natural or recombinant protein.

In another aspect, the invention provides a method of activating a biochemical pathway by exposing a cell to an artificial polypeptide or protein that is a modified form of a natural or recombinant protein activator of the biochemical pathway, wherein the modified form of the protein contains an unnatural amino acid.

In another aspect, the invention provides a method of inactivating a biochemical pathway, by exposing a cell to an artificial polypeptide or protein that is a modified form of a natural or recombinant protein is an inhibitor of the biochemical pathway, wherein the modified form of the protein contains an unnatural amino acid.

Therapeutic pathways that may be targeted for activation or inactivation include, but are not limited to, cell cycle regulatory pathways, development or differentiation pathways, metabolic pathways, one or more pathways involving any of the following molecular targets: oncogenes, tumor suppressors, nucleic acid repair proteins, vascular endothelial growth factor (VEGF) and its receptor (VEGFR); farnesyl transferase; matrix metalloproteinase; mTOR; COX-2; PDGF-R; protein kinase C; mitogen-activated protein kinase (MEK) ½; SRC kinase; histondeacetylase; small hypoxia-inducible factor HIF; aurora kinase; hedgehog; TGF-beta; TNF-alpha; human interleukin-1 receptor; endothelial growth factor; platelet surface membrane glycoprotein (GP) IIb/IIIa receptor in platelet aggregation; HIV fusion Protein; rapamycin (TOR); ribosomal protein S6 kinases (S6Ks); tumor suppressor proteins tuberous sclerosis 1 and 2 (TSC1 and 2); Ras-homolog enriched in brain (Rheb); TOR and phosphatidylinositol 3-kinase (PI3K); endothelin (ET); vasopressin; thyroid hormone and endothelin; phosphodiesterase type 5 (PDE 5); epidermal growth factor receptor (EGFR); vasopeptidase; interferon betas and glatiramer acetate; receptor and non-receptor tyrosine kinases (TKs); IL-2 receptor, Respiratory Syncytial Virus (RSV), thyroid stimulating hormone (TSH), somatostatin receptor, proteosome, keratinocyte growth hormone; CD20; alpha-4-Intgrin; pancreatic gastrin.

Accordingly, the invention provides a method of treating a patient by administering a therapeutically effective amount of an artificial polypeptide or protein to a patient in need thereof, wherein artificial polypeptide or protein is a modified form of a natural or recombinant protein is a therapeutic protein, and wherein the modified form of the protein contains an unnatural amino acid. In some embodiments, the artificial protein is therapeutically effective at a lower amount than the corresponding natural or recombinant therapeutic protein. In some embodiments, the corresponding natural or recombinant protein is an antibody, a hormone, an enzyme, a receptor, an antibiotic, a ligand, antigen, or other structural or functional protein. In certain embodiments, the patient may have a condition or disorder (e.g., an infection, cancer, diabetes, a neurodegenerative disorder, an immune system disorder, or other disease or condition).

Business Applications:

Aspects of the invention may be useful for reducing the time and/or cost of production, commercialization, and/or development of artificial polypeptides, proteins, and/or related therapeutic compositions. Accordingly, aspects of the invention relate to business methods that involve collaboratively (e.g., with a partner) or independently marketing one or more host cells, host cell lines, cell lysates, polypeptide preparations, artificial polypeptides, artificial proteins, nucleic acid cassettes, pharmaceutical preparations, or any combination of two or more thereof. For example, certain embodiments of the invention may involve marketing a library of different artificial polypeptides or proteins, a library of host cells that are capable of expressing different artificial polypeptide or proteins, and/or a library of nucleic acid cassettes that encode different artificial polypeptides or proteins.

Marketing may involve providing information relating to artificial polypeptide and protein compositions, production methods, and applications for potential customers or partners. Potential customers or partners may be, for example, companies in the pharmaceutical, biotechnology and agricultural industries, as well as academic centers and government research organizations or institutes. Business applications may involve generating revenue through sales and/or licenses of methods and/or compositions of the invention.

### EXAMPLES

### Example 1

### Modified tRNAs and tRNA Synthetases

The methods described herein may be used to produce synthetic genomes that may express a modified tRNA that inserts an amino acid upon exposure to a given codon that is not normally encoded by that codon. The amino acid may be a naturally occurring amino acid that is not normally associated with a given codon or the amino acid may be an unnatural amino acid. The term “naturally-occurring”, as applied to an object, refers to the fact that an object may be found in nature. For example, a polypeptide or polynucleotide sequence that is present in an organism (including bacteria) that may be isolated from a source in nature and which has not been intentionally modified by man in the laboratory is naturally-occurring or “wild type”. In an exemplary embodiment, the modified tRNA may be produced simply by changing the anticodon portion of the tRNA molecule itself. This modified tRNA molecule will still be charged with the same natural amino acid but will now insert this amino acid into a polypeptide chain in response to a different codon sequence. This type of modified tRNA may be constructed when the native tRNA synthetase that loads the tRNA does not interact or interacts minimally with the anticodon region during the loading process. In other embodiments, the tRNA synthetase may interact with the anticodon region of the tRNA molecule. In this case, a corresponding tRNA synthetase that is capable of loading the desired amino acid onto the tRNA must also be provided. Various references disclose methods for creating orthogonal tRNA/tRNA synthetase pairs that charge a desired amino acid onto a tRNA that recognizes a specific codon sequence. To date, over 100 noncoded amino acids (all ribosomally acceptable) have been reportedly introduced into proteins using various methods (see, for example, Schultz et al., J. Am. Chem. Soc., 103: 1563-1567, 1981; Hinsberg et al., J. Am. Chem. Soc., 104: 766-773, 1982; Pollack et al., Science, 242: 1038-1040, 1988; Nowak et al., Science, 268: 439-442, 1995; U.S. Patent Application Publication Nos. 2004/0053390; 2003/0143558; and 2003/0108885). These methods may be used for designing suitable altered amino acid tRNA synthetases (AARSs) that can efficiently charge a tRNA having a given anticodon with a desired amino acid.

A database of known tRNA genes and their sequences from a variety of organisms is publicly available (see e.g., http://rna.wustl.edu/GtRDB/). Similarly, a database of known aminoacyl tRNA synthetases has been published by Maciej Szymanski, Marzanna A. Deniziak and Jan Barciszewski, in Nucleic Acids Res. 29:288-290, 2001 (titled “Aminoacyl-tRNA synthetases database”). A corresponding website (http://rose.man.poznan.pl/aars/seq_main.html) provides details about all known AARSs from different species. For example, according to the database, the Isoleucyl-tRNA Synthetase for the radioresistant bacteria Deinococcus radiodurans (Accession No. AAF10907) has 1078 amino acids, and was published by White et al. in Science 286:1571-1577 (1999); the Valyl-tRNA Synthetase for mouse (Mus musculus) has 1263 amino acids (Accession No. AAD26531), and was published by Snoek M. and van Vugt H. in Immunogenetics 49: 468-470 (1999); and the Phenylalanyl-tRNA Synthetase sequences for human, Drosophila, S. pombe, S. cerevisiae, Candida albicans, E. coli, and numerous other bacteria including Thermus aquaticus ssp. thermophilus are also available. Similar information for other newly identified AARSs can be obtained, for example, by conducting a BLAST search using any of the known sequences in the AARS database as query against the available public (such as the non-redundant database at NCBI, or “r” or proprietary private databases.

Modifications of tRNA have been demonstrated to play several key roles in maintaining the tRNA's ability to faithfully decode an mRNA sequence. See, e.g., Qian, Q., et al., (1998) J Bacteriol 180(7):1808-13; Grosjean, H., et al., (1995) Biochimie 77:3-6; Esberg, B., et al., (1995) J Bacteriol 177(8):1967-75; and Grosjean, H., et al., (1998) “Modification and Editing of RNA,” American Society for Microbiology, Washington D.C., pp. 493-516. Furthermore, tRNA modifications have been implicated in full and proper translation of virulence genes in Shigella flexneri (see, e.g., Durand, J., et al., (1994) J Bacteriol 176(15):4627-34; Durand, J., et al., (1997) J Bacteriol 179(18):5777-82; and Durand, J., et al., (2000) Mol Microbiol 35(4):924-35) and in the plant pathogen Agrobacterium tumefaciens (see, e.g., Gray, J., et al. (1992) J Bacteriol 174(4):1086-9823). Modifications found at position 34 of the anticodon have been shown to change the coding capabilities of a particular tRNA by expanding or restricting the wobble rules at that position. For example, queosine (O) replaces a guanosine at position 34 in Tyr, H is, Asp, and Asn tRNAs and helps prevent misreading of the TAA/TAG STOP codons, and may prevent misreading of Gln, Lys, and Glu codons by restricting wobble. Alternatively, the lysidine modification at position 34 of the rare bacterial ileX tRNA changes its coding capacities from AUG to AUA (see, e.g., Muramatsu, T. et al., (1988) Nature 336(6195):179-81). Furthermore, modifications adjacent to the anticodon at position 37, including i6A and t6A, have been demonstrated to effect strand slipping and stop codon read through (see, e.g., Qian, Q., et al., (1998) J Bacteriol 180(7):1808-13; Esberg, B., et al., (1995) J Bacteriol 177(8):1967-75; and Miller, J., et al., (1976) Nuc Acids Res 3(5): 1185-201) and effects the fidelity of codon/anticodon interactions.

In certain embodiments, it may be desirable to utilize a modified tRNA, a modified tRNA synthetase, or a modified tRNA/tRNA synthetase pair. One of skill in the art will be able to produce such molecules based on the teachings herein. Modified tRNAs, tRNA synthetases and pairs thereof may be used to charge a tRNA having a given anticodon with a desired amino acid (either natural or unnatural). See e.g., U.S. Patent Application Publication No. 2003/0108885.

Methods for producing a modified tRNA synthetase are based on generating a pool of mutant synthetases from the framework of a wild-type synthetase, and then selecting for mutated RSs based on their specificity, for example, for an unnatural amino acid relative to the common twenty. The modified synthetase may be produced by mutating the synthetase, e.g., at the active site in the synthetase, at the editing mechanism site in the synthetase, at different sites by combining different domains of synthetases, or the like, and applying a positive and/or negative selection process (see e.g., U.S. Patent Application Publication No. 2003/0108885). In positive selection, suppression of a selector codon introduced at a nonessential position(s) of a positive marker allows cells to survive under positive selection pressure. In the presence of both natural and unnatural amino acids, survivors thus encode active synthetases charging the orthogonal suppressor tRNA with either a natural or unnatural amino acid. In the negative selection, suppression of a selector codon introduced at a nonessential position(s) of a negative marker removes synthetases with natural amino acid specificities. Survivors of the negative and positive selection encode synthetases that aminoacylate (charge) the orthogonal suppressor tRNA with unnatural amino acids only. These synthetases can then be subjected to further mutagenesis, e.g., DNA shuffling or other recursive mutagenesis methods. Of course, in other embodiments, the invention optionally can utilize different orders of steps to identify (e.g., RS, tRNA, pairs, etc.), e.g., negative selection/screening followed by positive selection/screening or vice verse or any such combinations thereof.

For example, a selector codon, e.g., an amber codon, is placed in a reporter gene, e.g., an antibiotic resistance gene, such as β-lactamase, with a selector codon, e.g., TAG. This is placed in an expression vector with members of the mutated RS library. This expression vector along with an expression vector with a target tRNA, e.g., a suppressor tRNA, are introduced into a cell, which is grown in the presence of a selection agent, e.g., antibiotic media, such as ampicillin. Only if the synthetase is capable of aminoacylating (charging) the suppressor tRNA with some amino acid does the selector codon get decoded allowing survival of the cell on antibiotic media. It should be understood that the term “gene” refers to a nucleic acid comprising an open reading frame encoding a polypeptide having exon sequences and optionally protein non-coding sequences, such as intron or intergenic sequences. The term “intron” refers to a DNA sequence present in a given gene which is not translated into protein and is generally found between exons.

Applying this selection in the presence of the unnatural amino acid, the synthetase genes that encode synthetases that have some ability to aminoacylate are selected away from those synthetases that have no activity. The resulting pool of synthetases can be charging any of the 20 naturally occurring amino acids or the unnatural amino acid. To further select for those synthetases that exclusively charge the unnatural amino acid, a second selection, e.g., a negative selection, is applied. In this case, an expression vector containing a negative selection marker and a target tRNA is used, along with an expression vector containing a member of the mutated RS library. This negative selection marker contains at least one selector codon, e.g., TAG. These expression vectors are introduced into another cell and grown without unnatural amino acids and, optionally, a selection agent, e.g., tetracycline. In the negative selection, those synthetases with specificities for natural amino acids charge the orthogonal tRNA, resulting in suppression of a selector codon in the negative marker and cell death. Since no unnatural amino acid is added, synthetases with specificities for the unnatural amino acid survive. For example, a selector codon, e.g., a stop codon, is introduced into the reporter gene, e.g., a gene that encodes a toxic protein, such as barnase. If the synthetase is able to charge the suppressor tRNA in the absence of unnatural amino acid, the cell will be killed by translating the toxic gene product. Survivors passing both selection/screens encode synthetases specifically charging the orthogonal tRNA with an unnatural amino acid.

In one embodiment, methods for producing at least one recombinant modified aminoacyl-tRNA synthetase (RS) include: (a) generating a library of mutant RSs derived from at least one aminoacyl-tRNA synthetase (RS) from a first organism; (b) selecting the library of mutant RSs for members that aminoacylate a target tRNA in the presence of an unnatural amino acid and a natural amino acid, thereby providing a pool of active mutant RSs; and, (c) negatively selecting the pool for active mutant RSs that preferentially aminoacylate the target tRNA in the absence of the unnatural amino acid, thereby providing the at least one modified recombinant RS; wherein the at least one recombinant modified RS preferentially aminoacylates the target tRNA with the unnatural amino acid. Optionally, more mutations are introduced by mutagenesis, e.g., random mutagenesis, recombination or the like, into the selected synthetase genes to generate a second-generation synthetase library, which is used for further rounds of selection until a mutant synthetase with desired activity is evolved. Recombinant modified RSs produced by the methods are included in the present invention.

The library of mutant RSs can be generated using various mutagenesis techniques known in the art. For example, the mutant RSs can be generated by site-specific mutations, random point mutations, in vitro homologous recombinant, chimeric constructs or the like. In one embodiment, mutations are introduced into the editing site of the synthetase to hamper the editing mechanism and/or to alter substrate specificity. Libraries of mutant RSs also include chimeric synthetase libraries, e.g., libraries of chimeric Methanococcus jannaschii/Escherichia coli synthetases. The domain of one synthetase can be added or exchanged with a domain from another synthetase, such as, for example, the CPI domain. See, e.g., Sieber, et al., Nature Biotechnology, 19:456-460 (2001). The chimeric library is screened for a variety of properties, e.g., for members that are expressed and in frame, for members that lack activity with a desired synthetase, and/or for members that show activity with a desired synthetase.

In one embodiment, the positive selection step includes: introducing a positive selection marker, e.g., an antibiotic resistance gene, or the like, and the library of mutant RSs into a plurality of cells, wherein the positive selection marker comprises at least one selector codon, e.g., an amber codon; growing the plurality of cells in the presence of a selection agent; selecting cells that survive in the presence of the selection agent by suppressing the at least one selector codon in the positive selection marker, thereby providing a subset of positively selected cells that contains the pool of active mutant RSs. Optionally, the selection agent concentration can be varied.

In one embodiment, negative selection includes: introducing a negative selection marker with the pool of active mutant RSs from the positive selection into a plurality of cells of a second organism, wherein the negative selection marker is an antibiotic resistance gene, e.g., a chloramphenicol acetyltransferase (CAT) gene, comprising at least one selector codon; and, selecting cells that survive in a 1st media supplemented with the unnatural amino acid and a selection agent, but fail to survive in a 2nd media not supplemented with the unnatural amino acid and the selection agent, thereby providing surviving cells with the at least one recombinant modified RS. Optionally, the concentration of the selection agent is varied.

In another embodiment, negatively selecting the pool for active mutant RSs includes: isolating the pool of active mutant RSs from the positive selection step (b); introducing a negative selection marker, wherein the negative selection marker is a toxic marker gene, e.g., a ribonuclease barnase gene, comprising at least one selector codon, and the pool of active mutant RSs into a plurality of cells of a second organism; and selecting cells that survive in a 1 st media not supplemented with the unnatural amino acid, but fail to survive in a 2nd media supplemented with the unnatural amino acid, thereby providing surviving cells with the at least one recombinant modified RS, wherein the at least one recombinant modified RS is specific for the unnatural amino acid. Optionally, the negative selection marker comprises two or more selector codons.

In one aspect, positive selection is based on suppression of a selector codon in a positive selection marker, e.g., a chloramphenicol acetyltransferase (CAT) gene comprising a selector codon, e.g., an amber stop codon, in the CAT gene, so that chloramphenicol can be applied as the positive selection pressure. In addition, the CAT gene can be used as both a positive marker and negative marker as describe herein in the presence and absence of unnatural amino acid. Optionally, the CAT gene comprising a selector codon is used for the positive selection and a negative selection marker, e.g., a toxic marker, such as a barnase gene comprising at least one or more selector codons, is used for the negative selection.

In another aspect, positive selection is based on suppression of a selector codon at nonessential position in the β-lactamase gene, rendering cells ampicillin resistant; and a negative selection using the ribonuclease barnase as the negative marker is used. In contrast to β-lactamase, which is secreted into the periplasm, CAT localizes in the cytoplasm; moreover, ampicillin is bacteriocidal, while chloramphenicol is bacteriostatic.

The recombinant modified RS can be further mutated and selected. In one embodiment, the methods for producing at least one recombinant modified aminoacyl-tRNA synthetase can further comprise: (d) isolating the at least one recombinant modified RS; (e) generating a second set of mutated RS derived from the at least one recombinant modified RS; and, (f) repeating steps (b) and (c) until a mutated RS is obtained that comprises an ability to preferentially aminoacylate the target tRNA. Optionally, steps (d)-(f) are repeated, e.g., at least about two times. In one aspect, the second set of mutated RS can be generated by mutagenesis, e.g., random mutagenesis, site-specific mutagenesis, recombination or a combination thereof.

The stringency of the selection steps, e.g., the positive selection step (b), the negative selection step (c) or both the positive and negative selection steps (b) and (c), in the above described-methods, optionally include varying the selection stringency. For example, because barnase is an extremely toxic protein, the stringency of the negative selection can be controlled by introducing different numbers of selector codons into the barnase gene. In one aspect of the present invention, the stringency is varied because the desired activity can be low during early rounds. Thus, less stringent selection criteria are applied in early rounds and more stringent criteria are applied in later rounds of selection.

Other types of selections can be used in the present invention for, e.g., modified RS, modified tRNA, and modified tRNA/RS pairs. For example, the positive selection step (b), the negative selection step (c) or both the positive and negative selection steps (b) and (c) can include using a reporter, wherein the reporter is detected by fluorescence-activated cell sorting (FACS). For example, a positive selection can be done first with a positive selection marker, e.g., chloramphenicol acetyltransferase (CAT) gene, where the CAT gene comprises a selector codon, e.g., an amber stop codon, in the CAT gene, which followed by a negative selection screen, that is based on the inability to suppress a selector codon(s), e.g., two or more, at positions within a negative marker, e.g., T7 RNA polymerase gene. In one embodiment, the positive selection marker and the negative selection marker can be found on the same vector, e.g., plasmid. Expression of the negative marker drives expression of the reporter, e.g., green fluorescent protein (GFP). The stringency of the selection and screen can be varied, e.g., the intensity of the light need to fluorescence the reporter can be varied. In another embodiment, a positive selection can be done with a reporter as a positive selection marker, which is screened by FACs, followed by a negative selection screen, that is based on the inability to suppress a selector codon(s), e.g., two or more, at positions within a negative marker, e.g., barnase gene.

Methods for producing a modified tRNA are also provided. For example, to change the codon specificity of the tRNA while preserving its affinity toward a desired RS, the methods include a combination of negative and positive selections with a mutant suppressor tRNA library in the absence and presence of the cognate synthetase, respectively. In the negative selection, a selector codon(s) is introduced in a marker gene, e.g., a toxic gene, such as barnase, at a nonessential position. When a member of the mutated tRNA library, e.g., derived from Methanococcus jannaschii, is aminoacylated by endogenous host, e.g., Escherichia coli synthetases (i.e., it is not orthogonal to the host, e.g., Escherichia coli synthetases), the selector codon, e.g., an amber codon, is suppressed and the toxic gene product produced leads to cell death. Cells harboring modified tRNAs or non-functional tRNAs survive. Survivors are then subjected to a positive selection in which a selector codon, e.g., an amber codon, is placed in a positive marker gene, e.g., a drug resistance gene, such a β-lactamase gene. These cells also contain an expression vector with a cognate RS. These cells are grown in the presence of a selection agent, e.g., ampicillin. tRNAs are then selected for their ability to be aminoacylated by the coexpressed cognate synthetase and to insert an amino acid in response to this selector codon. Cells harboring non-functional tRNAs, or tRNAs that cannot be recognized by the synthetase of interest are sensitive to the antibiotic. Therefore, tRNAs that: (i) are not substrates for endogenous host, e.g., Escherichia coli, synthetases; (ii) can be aminoacylated by the synthetase of interest; and (iii) are functional in translation survive both selections.

Methods of producing a modified tRNA include: (a) generating a library of mutant tRNAs derived from at least one tRNA, e.g., a suppressor tRNA, from a first organism; (b) negatively selecting the library for mutant tRNAs that are aminoacylated by an aminoacyl-tRNA synthetase (RS) from a second organism in the absence of a RS from the first organism, thereby providing a pool of mutant tRNAs; and, (c) selecting the pool of mutant tRNAs for members that are aminoacylated by an introduced RS, thereby providing at least one modified tRNA; wherein the at least one recombinant modified tRNA recognizes a selector codon and is not efficiently recognized by the RS from the second organism and is preferentially aminoacylated by the RS.

Libraries of mutated tRNA may be constructed. For example, mutations can be introduced at a specific position(s), e.g., at a nonconservative position(s), or at a conservative position, at a randomized position(s), or a combination of both in a desired loop of a tRNA, e.g., an anticodon loop, (D arm, V loop, T.psi.C arm) or a combination of loops or all loops. Chimeric libraries of tRNA are also included in the present invention. It should be noted that libraries of tRNA synthetases from various organism (e.g., microorganisms such as eubacteria or archaebacteria) such as libraries comprising natural diversity (such as libraries that comprise natural diversity (see, e.g., U.S. Pat. No. 6,238,884 to Short et al. and references therein, U.S. Pat. No. 5,756,316 to Schallenberger et al; U.S. Pat. No. 5,783,431 to Petersen et al; U.S. Pat. No. 5,824,485 to Thompson et al; and U.S. Pat. No. 5,958,672 to Short et al), are optionally constructed and screened for orthogonal pairs.

In one embodiment, negatively selecting the library for mutant tRNAs that are aminoacylated by an aminoacyl-tRNA synthetase (step (b) above) includes: introducing a toxic marker gene, wherein the toxic marker gene comprises at least one of the selector codons and the library of mutant tRNAs into a plurality of cells from the second organism; and, selecting surviving cells, wherein the surviving cells contain the pool of mutant tRNAs comprising at least one orthogonal tRNA or nonfunctional tRNA. For example, the toxic marker gene is optionally a ribonuclease barnase gene, wherein the ribonuclease barnase gene comprises at least one amber codon. Optionally, the ribonuclease barnase gene can include two or more amber codons. The surviving cells can be selected, e.g., by using a comparison ratio cell density assay.

In one embodiment, selecting the pool of mutant tRNAs for members that are aminoacylated by an introduced RS can include: introducing a positive selection marker gene, wherein the positive selection marker gene comprises a drug resistance gene, e.g., a β-lactamase gene, comprising at least one of the selector codons, e.g., a β-lactamase gene comprising at least one amber stop codon, the RS, and the pool of mutant tRNAs into a plurality of cells from the second organism; and, selecting surviving cells grown in the presence of a selection agent, e.g., an antibiotic, thereby providing a pool of cells possessing the at least one recombinant tRNA, wherein the recombinant tRNA is aminoacylated by the RS and inserts an amino acid into a translation product encoded by the positive marker gene, in response to the at least one selector codons. In another embodiment, the concentration of the selection agent is varied.

As described above for generating modified RS, the stringency of the selection steps can be varied. In addition, other selection/screening procedures, which are described herein, such as FACs, cell and phage display can also be used.

Methods for producing a recombinant modified tRNA include: (a) generating a library of mutant tRNAs derived from at least one tRNA, e.g., a suppressor tRNA, from a first organism; (b) selecting (e.g., negatively selecting) or screening the library for (optionally mutant) tRNAs that are aminoacylated by an aminoacyl-tRNA synthetase (RS) from a second organism in the absence of a RS from the first organism, thereby providing a pool of tRNAs (optionally mutant); and, (c) selecting or screening the pool of tRNAs (optionally mutant) for members that are aminoacylated by an introduced RS, thereby providing at least one recombinant modified tRNA; wherein the at least one recombinant modified tRNA recognizes a selector codon and is not efficiently recognized by the RS from the second organism and is preferentially aminoacylated by the RS. In some embodiments, the modified tRNA is optionally imported into a first organism from a second organism without the need for sequence modifications. In various embodiments, the first and second organisms are either the same or different and are optionally chosen from, e.g., prokaryotes (e.g., Methanococcus jannaschii, Methanobacteium thermoautotrophicum, Escherichia coli, Halobacterium, etc.), eukaryotes, mammals, fungi, yeasts, archaebacteria, eubacteria, plants, insects, protists, etc. Additionally, the recombinant tRNA is optionally aminoacylated by an unnatural amino acid, wherein the unnatural amino acid is biosynthesized in vivo either naturally or through genetic manipulation. The unnatural amino acid is optionally added to a growth medium for at least the first or second organism.

In one aspect, selecting (e.g., negatively selecting) or screening the library for (optionally mutant) tRNAs that are aminoacylated by an aminoacyl-tRNA synthetase (step (b)) includes: introducing a toxic marker gene, wherein the toxic marker gene comprises at least one of the selector codons (or a gene that leads to the production of a toxic or static agent or a gene essential to the organism wherein such marker gene comprises at least one selector codon) and the library of (optionally mutant) tRNAs into a plurality of cells from the second organism; and, selecting surviving cells, wherein the surviving cells contain the pool of (optionally mutant) tRNAs comprising at least one orthogonal tRNA or nonfunctional tRNA. For example, surviving cells can be selected by using a comparison ratio cell density assay.

In another aspect, the toxic marker gene can include two or more selector codons. In another embodiment of the methods, the toxic marker gene is a ribonuclease barnase gene, where the ribonuclease barnase gene comprises at least one amber codon. Optionally, the ribonuclease barnase gene can include two or more amber codons.

In one embodiment, selecting or screening the pool of (optionally mutant) tRNAs for members that are aminoacylated by an introduced RS can include: introducing a positive selection or screening marker gene, wherein the positive marker gene comprises a drug resistance gene (e.g., β-lactamase gene, comprising at least one of the selector codons, such as at least one amber stop codon) or a gene essential to the organism, or a gene that leads to detoxification of a toxic agent, along with the RS, and the pool of (optionally mutant) tRNAs into a plurality of cells from the second organism; and, identifying surviving or screened cells grown in the presence of a selection or screening agent, e.g., an antibiotic, thereby providing a pool of cells possessing the at least one recombinant tRNA, where the at least recombinant tRNA is aminoacylated by the RS and inserts an amino acid into a translation product encoded by the positive marker gene, in response to the at least one selector codons. In another embodiment, the concentration of the selection and/or screening agent is varied.

Methods for generating specific tRNA/RS pairs are also provided. Methods include: (a) generating a library of mutant tRNAs derived from at least one tRNA from a first organism; (b) negatively selecting or screening the library for (optionally mutant) tRNAs that are aminoacylated by an aminoacyl-tRNA synthetase (RS) from a second organism in the absence of a RS from the first organism, thereby providing a pool of (optionally mutant) tRNAs; (c) selecting or screening the pool of (optionally mutant) tRNAs for members that are aminoacylated by an introduced RS, thereby providing at least one recombinant tRNA. The at least one recombinant tRNA recognizes a selector codon and is not efficiency recognized by the RS from the second organism and is preferentially aminoacylated by the RS. The method also includes (d) generating a library of (optionally mutant) RSs derived from at least one aminoacyl-tRNA synthetase (RS) from a third organism; (e) selecting or screening the library of mutant RSs for members that preferentially aminoacylate the at least one recombinant tRNA in the presence of an unnatural amino acid and a natural amino acid, thereby providing a pool of active (optionally mutant) RSs; and, (f) negatively selecting or screening the pool for active (optionally mutant) RSs that preferentially aminoacylate the at least one recombinant tRNA in the absence of the unnatural amino acid, thereby providing the at least one specific tRNA/RS pair, wherein the at least one specific tRNA/RS pair comprises at least one recombinant RS that is specific for the unnatural amino acid and the at least one recombinant tRNA. Specific tRNA/RS pairs produced by the methods are included. For example, the specific tRNA/RS pair can include, e.g., a mutRNATyr-mutTyrRS pair, such as a mutRNATyr-SS12TyrRS pair, a mutRNALeu-mutLeuRS pair, a mutRNAThr-mutThrRS pair, a mutRNAGlu-mutGluRS pair, or the like. Additionally, such methods include wherein the first and third organism are the same.

Methods for selecting a tRNA-tRNA synthetase pair for use in an in vivo translation system of a second organism are also included in the present invention. The methods include: introducing a marker gene, a tRNA and an aminoacyl-tRNA synthetase (RS) isolated or derived from a first organism into a first set of cells from the second organism; introducing the marker gene and the tRNA into a duplicate cell set from a second organism; and, selecting for surviving cells in the first set that fail to survive in the duplicate cell set or screening for cells showing a specific screening response that fail to give such response in the duplicate cell set, wherein the first set and the duplicate cell set are grown in the presence of a selection or screening agent, wherein the surviving or screened cells comprise the orthogonal tRNA-tRNA synthetase pair for use in the in the in vivo translation system of the second organism. In one embodiment, comparing and selecting or screening includes an in vivo complementation assay. The concentration of the selection or screening agent can be varied.

In certain embodiments, tRNAs may be used to incorporate unnatural amino acids into a protein. Exemplary unnatural amino acids are described, for example, in U.S. Patent Application Publication No. 2003/0108885 and include, for example, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine. Additionally, other examples optionally include (but are not limited to) an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkyl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photo caged amino acid; a photoisomerizable amino acid; a biotin or biotin-analogue containing amino acid; a glycosylated or carbohydrate modified amino acid; a keto containing amino acid; an amino acid comprising polyethylene glycol; an amino acid comprising polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline.

For example, many unnatural amino acids are based on natural amino acids, such as tyrosine, glutamine, phenylalanine, and the like. Tyrosine analogs include para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, or the like. In addition, multiply substituted aryl rings are also contemplated. Glutamine analogs of the invention include, but are not limited to, α-hydroxy derivatives, β-substituted derivatives, cyclic derivatives, and amide substituted glutamine derivatives. Example phenylalanine analogs include, but are not limited to, meta-substituted phenylalanines, wherein the substituent comprises a hydroxy group, a methoxy group, a methyl group, an allyl group, an acetyl group, or the like. Specific examples of unnatural amino acids include, but are not limited to, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In certain embodiments, unnatural amino acids may be selected or designed to provide additional characteristics unavailable in the twenty natural amino acids. For example, unnatural amino acid are optionally designed or selected to modify the biological properties of a protein, e.g., into which they are incorporated. For example, the following properties are optionally modified by inclusion of an unnatural amino acid into a protein: toxicity, biodistribution, solubility, stability, e.g., thermal, hydrolytic, oxidative, resistance to enzymatic degradation, and the like, facility of purification and processing, structural properties, spectroscopic properties, chemical and/or photochemical properties, catalytic activity, redox potential, half-life, ability to react with other molecules, e.g., covalently or noncovalently, and the like.

### Example 2

### Hierarchical Assembly Methods

Host organisms containing a threshold number of replaced codons and/or other genetic changes such as modified tRNA synthetases may be produced via hierarchical assembly of large genomic regions containing the modified codons and/or gene sequences. Synthesis of large, even genome sized polynucleotide constructs, may be produced by a variety of methods available to one of skill in the art based on the disclosure herein. For example, in one embodiment, a synthetic genome may be produced by substituting into the organism's genome a plurality of synthetic DNA segments homologous with the native sequence but implementing the substituted sequences. In another embodiment, an entire genome may be synthesized and then used to replace the naturally occurring genome in the cell. For example, a genome may be synthesized in a bacterial cell and then transferred to another cell, e.g., another bacterial cell, yeast, plant, or eukaryotic cell (including a mammalian cell), by means of conjugation. Both of these techniques require the production of long molecules of DNA having high fidelity. Such DNA molecules may be produced using a variety of methods such as high throughput oligonucleotide assembly techniques described in Zhou et al. Nucleic Acids Research, 32: 5409-5417 (2004); Richmond et al. Nucleic Acids Research 32: 5011-5018 (2004); Tian et al. Nature 432: 1050-1054 (2004); and Carr et al. Nucleic Acids Research 32: e162 (2004). For example, oligonucleotides having complementary, overlapping sequences may be synthesized on a chip and then eluted off. The oligonucleotides then are induced to self assemble based on hybridization of the complementary regions.

In one aspect, the invention provides a method for hierarchical assembly of very large, including genome sized, polynucleotide constructs. The goal of hierarchical DNA assembly is to reduce the number of steps required to construct large synthetic DNA (e.g. chromosomes) from N steps to as little as log 2(N). This could be accomplished by a series of DNA preps and electroporations, but as the DNA pieces get longer than about 50,000 bp (e.g., 50 kbp), DNA fragility becomes a factor. Bacterial conjugation allows transfer of up to 5 Mbp from a bacterium to another bacterium (Li M Z and Elledge S J. Nat. Genet. (2005) 37(3): 311-9), or from a bacterium to yeast, plant or mammalian cells (Waters V L Nat. Genet. (2001) 29(4): 375-6).

For purposes of comparison, a serial/sequential assembly approach for producing a large polynucleotide construct would involve repeated replacements of portions of the genome or plasmid sequence one at a time. For example, replacement of all sequences of an organism's genome may be carried out by serially substituting into the genome synthetic DNA segments having alternating selectable markers. For example, the entire genome of E. coli may be synthesized by introducing 48 synthetic DNA segments of approximately 100 kb into a cell having, for example, λ-red recombinase. Each 100 kb segment contains, for example, either a kanamycin resistant gene (Kan^{R}) or a tetracycline resistant gene (tet^{R}) toward the left-end of the 100 kb fragment. A 100 kb segment that contains, for example, a Kan^{R} is first introduced into the genome and the cells are selected for kanamycin resistance. In a next step, a second 100 kb segment that contains a tet^{R} gene is introduced into the cell. The tet^{R} 100 kb segment is targeted so that its right-end destroys the Kan^{R} gene of the first segment and its left-end introduces a tet^{R} gene. Cells that are tetracycline resistant and kanamycin sensitive will have the first and second segments properly incorporated into the genome. Subsequent 100 kb segments containing alternating Kan^{R}and tet^{R} markers may be introduced into the genome to sequentially replace segments of the genome. In this embodiment, for example, 48 segments of 100 kb may be sequentially introduced with alternating selectable markers to synthesize the entire E. coli genome.

The serial approach requires a separate and sequential step for introduction of each subsequent polynucleotide construct and therefore is very time consuming. For example, the serial approach may be further illustrated with respect to creating a synthetic E. coli genome. Replacement of the entire genome requires about 48 polynucleotide constructs (e.g., each segment being about 100 kb long for a 4.8 Mbp genome like E. coli) with alternating markers 2 and 3. Recombining these segments serially into the genome would take 48 stages at about 2 days per stage (e.g., one strain having increasing portions of its genome replaced). For illustration purposes this process is represented by 6 polynucleotide segments and hence 6 stages below. The dashes “-” are simply alignment symbols and do not represent any base pairs. The starting genome (or plasmid polynucleotide) sequence is shown in capital letters and the goal genome (or plasmid polynucleotide sequence) is shown in lower cases letters.

Starting and Goal Genomes:

| | |
|---|---|
| Initial genome: | ABCDEFGHIJKLMNOPQRSTUVWXYZ |
| Goal genome: | abcdefghijklmnopqrstuvwxy2z |

It should be noted that since the polynucleotides (e.g., genomes or plasmids) are circular, the above goal genome could also be represented, for example, as bcdefghijklmnopqrstuvwxy2za. In this example, selectable genes are referred to by numbers 2 and 3, for example, 2 may be chloramphenicol resistance and 3 may be kanamycin resistance. In each case, one of the markers may be selected for to obtain the desired construct.

Sequential Assembly Starting Constructs:

```
abcde3fg
      de-fghi2jk
                hi-jklm3no
                          lm-nopq2rs
                                    pq-rstu3vw
                                              tu-vwxyz2abc
```

Each segment is sequentially introduced into the genome as illustrated below:

Abcde3fGHIJKLMNOPQRSTUVWXYZ
Abcdefghi2jKLMNOPQRSTUVWXYZ
Abcdefqhijklm3nOPQRSTUVWXYZ
Abcdefqhijklmnopq2rSTUVWXYZ
Abcdefghijklmnopqrstu3vWXYZ
-bcdefghijklmnopqrstuvwxyz2a (final product)

The hierarchical assembly approach may reduce this assembly procedure to only seven stages. Each stage involves multiple strains having one or more segments of their genome replaced with a synthetic polynucleotide construct. For example, at the first stage, 48 different strains each containing a single synthetic polynucleotide construct replacing a portion of the genome are produced. These 48 strains are then combined pairwise to produce 24 strains each comprising two segments of their genome replaced by synthetic polynucleotide constructs. This process is repeated iteratively until the entire genome has been substituted, e.g., the 24 strains are combined pairwise to produce 12 strains having four synthetic segments each, the 12 strains are combined pairwise to produce 6 strains having 8 synthetic segments each, and so on, thereby producing, 3, 2 and finally 1 strain having the entire genome replaced with synthetic polynucleotide segments. The assembly may be facilitated by bacterial conjugation which permits transfer of large DNA segments from one cell to another. Alternatively, in certain embodiments, the DNA segments may be introduced into the cells by a transfection procedure (e.g., electroporation, etc.).

Conjugative transfer of DNA from a bacterium requires trans-acting proteins (e.g. tra genes) and a cis-acting nicking site (origin of transfer or oriT). After nicking the donor genome (or plasmid), a strand-displacing polymerase pushes a DNA copy (starting at the nick primer) into the recipient cell. A variety of oriT sites may be used in conjunction with the methods described herein. For example, ColE1 oriT can be as small as 22 bp (Heeb S, et al. Mol Plant Microbe Interact. (2000) 13(2): 232-7) while oriT sites for the broad host range plasmid RK2 is about 250 bp (Guiney D G, et al. Plasmid. (1988) 20(3): 259-65). Other compatible oriT sites that may be used in accordance with the methods described herein include IncPalpha, F, and R64 (IncI). Some conjugative systems will not mate with the same mating type efficiently. Accordingly, in certain embodiments, the donor sequences may include tra genes (e.g., traF, traG and traJ) which are transferred into the recipient cell that does not contain any tra genes. In an exemplary embodiment, the tra genes may be provided under the control of an inducible or repressible promoter. Additionally, if the oriT is destroyed in the process of transfer, a new oriT site may be made available in the recipient cell for the next round of assembly.

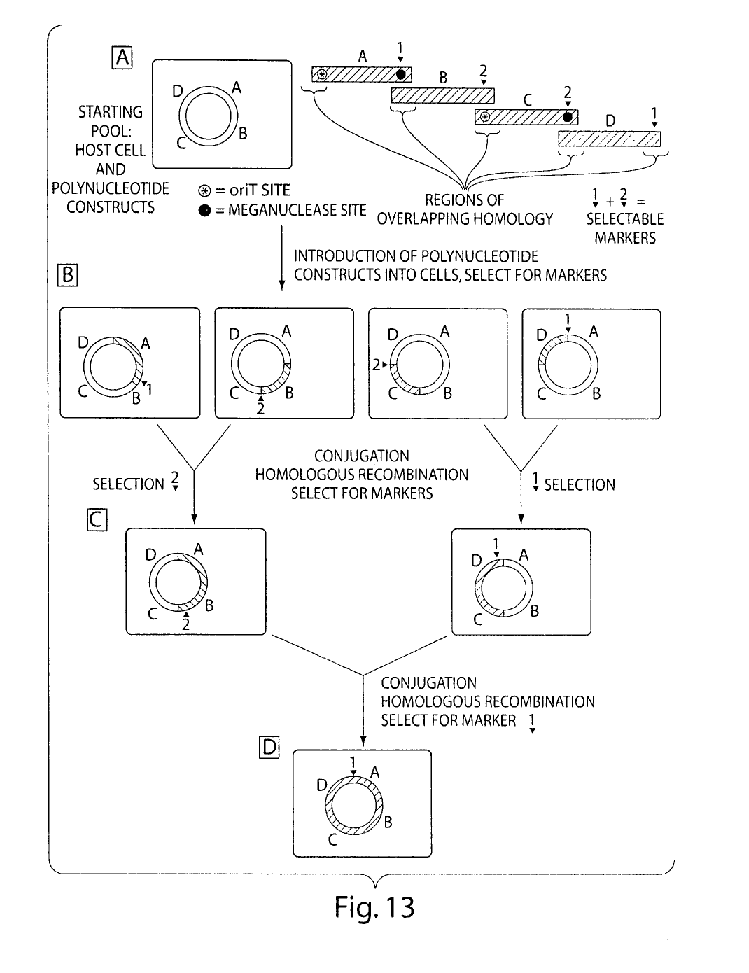

The hierarchical assembly process may be illustrated by the following example and also with reference to FIG. 13. For illustration purposes, the process is represented by 6 polynucleotide segments that require only 3 stages rather than 6 stages as was required for the serial assembly approach. Each segment represents a ˜100 kb construct. The markers (e.g., 2 and 3) are arranged in a different order and do not alternate with each sequential segment. Additionally, as shown below, some of the polynucleotide segments may comprise oriT sites (illustrated as 4, 6, and 8) and meganuclease sites (illustrated as 5, 7, and 9). Any type of meganuclease may be used in association with the methods described herein, including, for example, I-SceI, I-DmoI, I-Crel, and E-DreI (Chevalier B S, et al. Mol. Cell. (2002) 10(4): 895-905; Posfai G, et al. Nucleic Acids Res. (1999) 27(22): 4409-15). In an exemplary embodiment, the meganuclease used does not create a double stranded break in the genome of the host cell being used for assembly. The oriT nicking and meganuclease sites direct the recombination machinery by creating recombinogenic ends. At each stage, one of the selectable markers may be used to obtain the desired product.

Heirarchical Assembly Starting Constructs:

| | |
|---|---|
| 4abcde3fg5 | |
| de-fghi2jk | |
| 6hi-jklm2no7 | (switched |
| | 2 & 3) |
| lm-nopq3rs | (switched |
| | 2 & 3) |
| 8pq-rstu3vw9 | |
| tu-vwxyz2abc | |

The starting constructs are each introduced into separate cells which are then combined pairwise to build up larger polynucleotide constructs as follows:

| | |
|---|---|
| Abcde3fGHI-JKLM-NOPQ-RSTU-VWXYZ | Donor-top |
| ABCDe-fghi2jKLM-MOPQ-RSTU-VWXYZ | Recipient-top |
| ABCDE-FGHi-jklm2nOPQ-RSTU-VWXYZ | Donor-mid |
| ABCDE-FGHI-JKLm-nopq3rSTU-VWXYZ | Recipient-mid |
| ABCDE-FGHI-JKLM-NOPq-rstu3vWXYZ | Donor-low |
| --CDE-FGHI-JKLM-NOPQ-RSTu-vwxyz2ab | Recipient-low |

Following pairwise combinations, three new strains are produced having the genomes as illustrated below:

| | |
|---|---|
| Abcde-fghi2jKLM-NOPQ-RSTU-VWXYZ | New Donor (tops, select for 2 against 3) |
| ABCDE-FGHi-jklm-nopq3rSTU-VWXYZ | Recipient (mids, select for 3 against 2) |
| --CDE-FGHI-JKLM-NOPq-rstu-vwxyz2ab | Waiting (lows, select for 2 against 3) |

The new genomes are then combined pairwise to produce the two genomes as illustrated below:

| | |
|---|---|
| Abcde-fghi-jklm-nopq3rSTu-VWXYZ | New-new Donor (select for 3 against 2) |
| --CDE-FGHI-JKLM--NOPq-rstu-vwxyz2ab | Recipient |

The two strains having the genomes illustrated above may then be combined to produce the final desired product as follows:

| | |
|---|---|
| bcde-fghi-jklm-nopq-rstu-vwxyz2a | (select for 2 against 3) |

This process of hierarchical assembly significantly reduces the time and effort required to produce the final product as compared to the serial/sequential assembly approach. For example, the sequential assembly approach would require approximately 48 stages each taking approximately 2 days, or a total time of about 96 days for producing a synthetic E. coli genome. In contrast, the hierarchical assembly methods provided herein would only require about 7 stages of about 2 days each, or approximately 14 days total. Additionally, cellular conjugation as a means of introducing DNA segments into a cell is faster than electroporations. Therefore, if selectable markers are chosen that permit both positive and negative selections, then the whole process could be done in liquid cultures (e.g. thyA, galK, and/or supF) and the 7 stages of assembly could be completed in as little as 2 days total time. In exemplary embodiments, such hierarchical assembly methods could be carried out in highly-parallel multi-well plates allowing many such genome constructions simultaneously.

Each of the genome replacements must produce a viable intermediate. In certain embodiments, each of the 100 kb segments may be simultaneously introduced into cells and pre-tested for viable intermediates. If any of the 100 kb segments produces an inviable intermediate, one or more segments may be altered until a complete set of viable intermediates is generated.

The hierarchical assembly methods disclosed herein are further graphically illustrated in FIG. 13. Part A shows the starting materials including a cell with an initial genome (empty) and variety of polynucleotide constructs that together comprise the sequences of the synthetic genome (hatched; labeled A, B, C, and D). The polynucleotide constructs comprise selectable markers (labeled 1 and 2) and some of them comprise oriT sites (*) and meganuclease cleavage sites (•). Additionally, each polynucleotide construct comprises overlapping regions of sequence homology at each termini (e.g., both termini of each polynucleotide construct have overlapping homology with one other polynucleotide construct). Each polynucleotide construct is separately introduced into a cell, for example, by electroporation or any other method for cellular transformation. The polynucleotide segments then integrate into the host cell genome by homologous recombination. In certain embodiments, it may be desirable to utilize a host cell that is overexpressing a recombinase and/or comprises a recombinase under the control of an inducible/repressible promoter. Exemplary recombinase systems include, for example, RedE and RecT proteins (from E. coli) or Redα, Redβ and Gam proteins (from lambda). Examples of inducible promoters include, for example, promoters under lac control or rharnose control (rhaB promoter). The products produced by the introduction of the polynucleotide constructs are a plurality of cells comprising a single integrated synthetic segment replacing the wild-type sequence at that location (see FIG. 13B). In certain embodiments, the polynucleotide segments may integrate into the genome of the cell while in other embodiments the polynucleotide segments may integrate into an extrachromosomal nucleotide construct such as a plasmid or artificial chromosome.

Assembly into larger polynucleotide constructs, or whole genome replacement, is achieved by repeated rounds of conjugation and integration (see FIGS. 13C and D). For example, cells comprising synthetic segments with homologous overlapping termini are mixed together and the DNA from one cell (the donor) is transferred into another cell (the recipient cell). The second synthetic segment is then integrated into the appropriate location in the genome by homologous recombination (e.g., as aligned by the overlapping homologous regions on the synthetic segments). The desired product may be selected using the appropriate selectable marker (see FIG. 13C). This process of conjugation and recombination is repeated until the desired polynucleotide construct (or synthetic genome) has been constructed. In alternative embodiments, additional segments of synthetic DNA may be introduced into the cells by transfection techniques, such as electroporation, rather than conjugation. In yet other embodiments, various combinations of electroporation and conjugation may be used. The final product may be transferred into a desired cell by conjugation.

In one embodiment, polynucleotide constructs or segments suitable for assembly of larger polynucleotide constructs such as synthetic genomes may be produced, for example, using a nucleic acid array for the direct fabrication of DNA or other nucleic acid molecules of any desired sequence and of indefinite length. Sections or segments of the desired nucleic acid molecule are fabricated on an array, such as by way of a parallel nucleic acid synthesis process using an array synthesizer instrument. After the synthesis of the segments, the segments are assembled to make the desired molecule. In essence the technique permits the quick easy and direct synthesis of nucleic acid molecule for any purpose in a simple and quick synthesis process.

A salient feature of this technique relates to use of low-purity arrays, e.g., arrays having features of less than 10 percent purity with respect to any given nucleic acid sequence. The utility of the low-purity arrays arises from the ability to correct errors occurring in the assembled constructs.

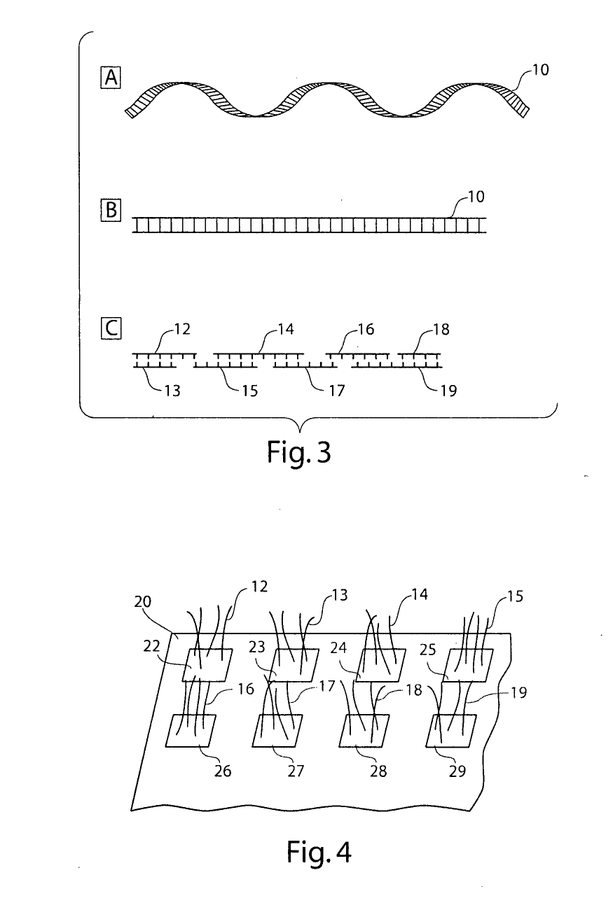

An illustration of the direct fabrication of a relatively simple DNA molecule is described in the figures. In FIG. 3, at 10, a double stranded DNA molecule of known sequence is illustrated. That same molecule is illustrated in both the familiar double helix shape in FIG. 3A, as well as in an untwisted double stranded linear shape shown in FIG. 3B. Assume, for purposes of this illustration, that the DNA molecule is broken up into a series of overlapping single smaller stranded DNA molecule segments, indicated by the reference numerals 12 through 19 in FIG. 3C. The even numbered segments are on one strand of the DNA molecule, while the odd numbered segments form the opposing complementary strand of the DNA molecule. The single stranded molecule segments can be of any reasonable length, but can be conveniently all of the same length which, for purposes of this example, might be 100 base pairs in length. Since the sequence of the molecule 10 of FIG. 3A is known, the sequence of the smaller DNA segments 12 through 19 can be defined simply be breaking the larger sequence into overlapping sequences each of 100 base pairs. In the normal nomenclature of the art, the DNA sequences on the microarray are sometimes referred to as probes because of the intended use of the DNA sequences to probe biological samples. Here these same sequences are referred to as DNA segments, also because of the intended use of these sequences.

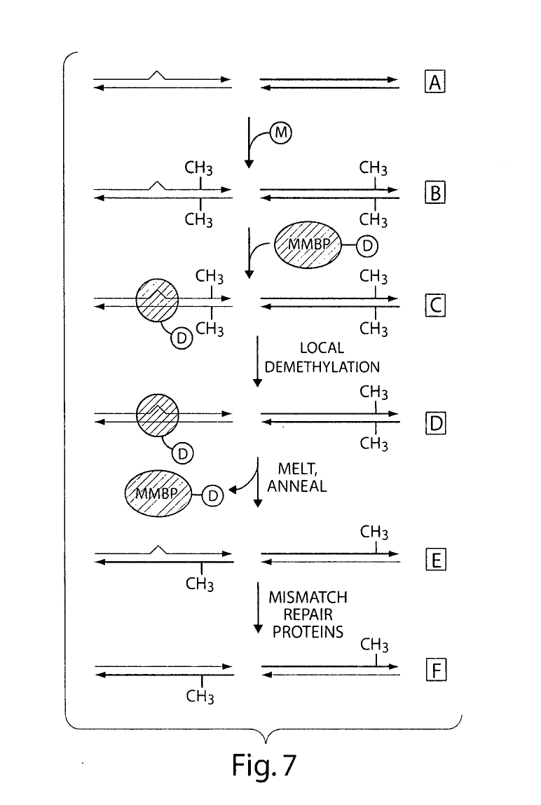

The information about the sequence of the segments 12-19 is then used to construct a new totally fabricated DNA molecule. This process is initiated by constructing a microarray of single stranded DNA segments on a common substrate. This process is illustrated in FIG. 4. Each of the single stranded segments 12 through 19 is constructed in a single cell, or feature, of a DNA microarray indicated at 20. Each of the DNA segments is fabricated in situ in a corresponding feature indicated by reference numbers 22 through 29. Such a microarray is preferably constructed using a maskless array synthesizer (MAS), as for example of the type described in published PCT patent application WO99/42813 and in corresponding U.S. Pat. No. 6,375,903, the disclosure of each of which is herein incorporated by reference. Other examples are known of maskless instruments which can fabricate a custom DNA microarray in which each of the features in the array has a single stranded DNA molecule of desired sequence. The preferred type of instrument is the type shown in FIG. 7 of U.S. Pat. No. 6,375,903, based on the use of reflective optics. It is a desirable and useful advantage of this type of maskless array synthesizer in that the selection of the DNA sequences of the single stranded DNA segments is entirely under software control. Since the entire process of microarray synthesis can be accomplished in only a few hours, and since suitable software permits the desired DNA sequences to be altered at will, this class of device makes it possible to fabricate microarrays including DNA segments of different sequence every day or even multiple times per day on one instrument. The differences in DNA sequence of the DNA segments in the microarray can also be slight or dramatic, it makes no different to the process. The usual use of such microarrays is to perform hybridization test on biological samples to test for the presence or absence of defined nucleic acids in the biological samples. Here, a much different use for the microarray is contemplated.

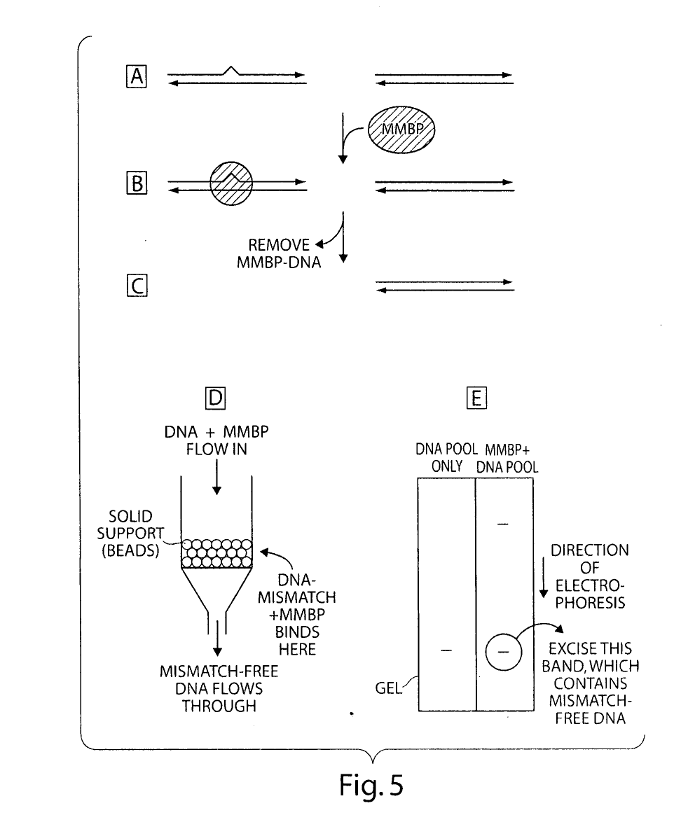

The MAS instrument may be used in the form it would normally be used to make microarrays for hybridization experiments, but it may also be adapted to have features specifically adapted for this application. For example, it may be desirable to substitute a coherent light source, i.e. a laser, for the light source shown in FIG. 5 of the above-mentioned U.S. Pat. No. 6,375,903. If a laser is used as the light source, a beam expanded and scatter plate may be used after the laser to transform the narrow light beam from the laser into a broader light source to illuminate the micromirror arrays used in the maskless array synthesizer. It is also envisioned that changes may be made to the flow cell in which the microarray is synthesized. In particular, it is envisioned that the flow cell can be compartmentalized, with linear rows of array elements being in fluid communication with each other by a common fluid channel, but each channel being separated from adjacent channels associated with neighboring rows of array elements. During microarray synthesis, the channels all receive the same fluids at the same time. After the DNA segments are separated from the substrate, the channels serve to permit the DNA segments from the row of array elements to congregate with each other and begin to self-assemble by hybridization. This alternative will also be discussed further below.

Once the fabrication of the DNA microarray is completed, the single stranded DNA molecule segments on the microarray are then freed or eluted from the substrate on which they were constructed. The particular method used to free the single stranded DNA segments is not critical, several techniques being possible. The DNA segment detachment method most preferred is a method which will be referred to here as the safety-catch method. Under the safety-catch approach, the initial starting material for the DNA strand construction in the microarray is attached to the substrate using a linker that is stable under the conditions required for DNA strand synthesis in the MAS instrument conditions, but which can be rendered labile by appropriate chemical treatment. After array synthesis, the linker is first rendered labile and then cleaved to release the single stranded DNA segments. The preferred method of detachment for this approach is cleavage by light degradation of a photo-labile attachment group. The oligos may be released all at once across the array field or one region at a time to promote correct intended hybridization, a technique particularly helpful in the synthesis of large sequences containing repeats.

These synthetic single stranded DNA segments are suspended in a solution under conditions which favor the hybridization of single stranded DNA strands into double stranded DNA. Under these conditions, the single stranded DNA segments will automatically begin to assemble the desired larger complete DNA sequence. This occurs because, for example, the 3′ half of the DNA segment 12 will either preferentially or exclusively hybridize to the complementary half of the DNA segment 13. This is because of the complementary nature of the sequences on the 3′ half of the segment 12 and the sequence on the 5′ half of the segment 13. The half of the segment 13 that did not hybridize to the segment 12 will then, in turn, hybridize to the 3′ half of the segment 14. This process will continue spontaneously for all of the segments freed from the microarray substrate. By this process, a DNA assembly similar to that indicated in FIG. 3C is created. By joining the aligned single stranded DNA molecules to each other, as can be done with a DNA ligase, the DNA molecule 10 of FIG. 3A is completed. The number of copies of the molecule created will be proportional to the copy number of identical segments synthesized in each of the features in the microarray 20. It may also be desirable to assist the assembly of the completed DNA molecule by performing one of a number of types of sub-assembly reactions. Several alternatives for such reactions are described below.

As the molecule length grows, conventional methods of error-reduction become prohibitively cumbersome and costly. Set forth below are tools for dramatically reducing errors in large-scale gene synthesis.

Biological organisms have means to detect errors in their own DNA sequences, as well as repair them. One component of this system is a mismatch binding protein which can detect short regions of DNA containing a mismatch, a region where the two DNA strands are not perfectly complementary to each other. Mismatches can be the result of a point mutation, deletion, insertion, or chemical modification. For the purpose of this invention, a mismatch includes base pairs of opposing strands with sequence A-A, C-C, T-T, G-G, A-C, A-G, T-C, T-G, or the reverse of these pairs (which are equivalent, i.e. A-G is equivalent to G-A), a deletion, insertion, or other modification to one or more of the bases. The mismatch binding proteins (MMBPs) have been used commercially for the detection of mutations and genetic differences within a population (SNP genotyping), but not for the purpose of error control in designed sequences.

In various embodiments, mismatch binding proteins can be used to control the errors generated during oligonucleotide synthesis, gene assembly, and the construction of nucleic acids of different sizes. (Though biological systems use this function when synthesizing DNA, it requires the presence of a template strand. For de novo synthesis, as employed by this technique, one is starting by definition without a template.)

When attempting to produce a desired DNA molecule, a mixture typically results containing some correct copies of the sequence, and some containing one or more errors. But if the synthetic oligonucleotides are annealed to their complementary strands of DNA (also synthesized), then a single error at that sequence position on one strand will give rise to a base mismatch, causing a distortion in the DNA duplex. These distortions can be recognized by a mismatch binding protein. (One example of such a protein is MutS from the bacterium Escherichia coli.) Once an error is recognized, a variety of possibilities exist for how to prevent the presence of that error in the final desired DNA sequence.

When using pairs of complementary DNA strands for error recognition, each strand in the pair may contain errors at some frequency, but when the strands are annealed together, the chance of errors occurring at a correlated location on both strands is very small, with an even smaller chance that such a correlation will produce a correctly matched Watson-Crick base pair (e.g. A-T, G-C). For example, in a pool of 50-mer oligonucleotides, with a per-base error rate of 1%, roughly 60% of the pool (0.9950) will have the correct sequence, and the remaining forty percent will have one or more errors (primarily one error per oligonucleotide) in random positions. The same would be true for a pool composed of the complementary 50-mer. After annealing the two pools, approximately 36% (0.62) of the DNA duplexes will have correct sequence on both strands, 48% (2×0.4×0.6) will have an error on one strand, and 16% (0.42) will have errors in both strands. Of this latter category, the chance of the errors being in the same location is only 2% ( 1/50) and the chance of these errors forming a Watson-Crick base pair is even less (⅓× 1/50). These correlated mismatches, which would go undetected, then comprise 0.11% of the total pool of DNA duplexes (16×⅓× 1/50). Removal of all detectable mismatch-containing sequences would thus enrich the pool for error-free sequences (i.e. reduce the proportion of error-containing sequences) by a factor of roughly 200 (0.6/0.4 originally for the single strands vs. 0.36/0.0011 after mismatch detection and removal). Furthermore, the remaining oligonucleotides can then be dissociated and re-annealed, allowing the error-containing strands to partner with different complementary strands in the pool, producing different mismatch duplexes. These can also be detected and removed as above, allowing for further enrichment for the error-free duplexes. Multiple cycles of this process can in principle reduce errors to undetectable levels. Since each cycle of error control may also remove some of the error-free sequences (while still proportionately enriching the pool for error-free sequences), alternating cycles of error control and DNA amplification can be employed to maintain a large pool of molecules.

In one embodiment, the number of errors detected and corrected may be increased by melting and reannealing a pool of DNA duplexes prior to error correction. For example, if the DNA duplexes in question have been amplified by a technique such as the polymerase chain reaction (PCR) the synthesis of new (perfectly) complementary strands would mean that these errors are not immediately detectable as DNA mismatches. However, melting these duplexes and allowing the strands to re-associate with new (and random) complementary partners would generate duplexes in which most errors would be apparent as mismatches, as described above.

Many of the methods described below can be used together, applying error-reducing steps at multiple points along the way to produce a long nucleic acid molecule. Error reduction can be applied to the first oligonucleotide duplexes generated, then for example to intermediate 500-mers or 1000-mers, and then even to larger full length nucleic acid sequences of 10,000-mers or more. In an exemplary embodiment, the methods described herein may be used to produce the entire genome of an organism optionally incorporating specific modifications into the sequence at one or more desired locations.

FIG. 5 illustrates an exemplary method for removing sequence errors using mismatch binding proteins. An error in a single strand of DNA causes a mismatch in a DNA duplex. A mismatch recognition protein (MMBP), such as a dimer of MutS, binds to this site on the DNA. As shown in FIG. 5A, a pool of DNA duplexes contains some duplexes with mismatches (left) and some which are error-free (right). The 3′-terminus of each DNA strand is indicated by an arrowhead. An error giving rise to a mismatch is shown as a raised triangular bump on the top left strand. As shown in FIG. 5B, a MMBP may be added which binds selectively to the site of the mismatch. The MMBP-bound DNA duplex may then be removed, leaving behind a pool which is dramatically enriched for error-free duplexes (FIG. 5C). In one embodiment, the DNA-bound protein provides a means to separate the error-containing DNA from the error-free copies (FIG. 5D). The protein-DNA complexes can be captured by affinity of the protein for a solid support functionalized, for example, with a specific antibody, immobilized nickel ions (protein is produced as a his-tag fusion), streptavidin (protein has been modified by the covalent addition of biotin) or other such mechanisms as are common to the art of protein purification. Alternatively, the protein-DNA complex is separated from the pool of error-free DNA sequences by a difference in mobility, for example, using a size-exclusion column chromatography or by electrophoresis (FIG. 5E). In this example, the electrophoretic mobility in a gel is altered upon MMBP binding: in the absence of MMBP all duplexes migrate together, but in the presence of MMBP, mismatch duplexes are retarded (upper band). The mismatch-free band (lower) is then excised and extracted.

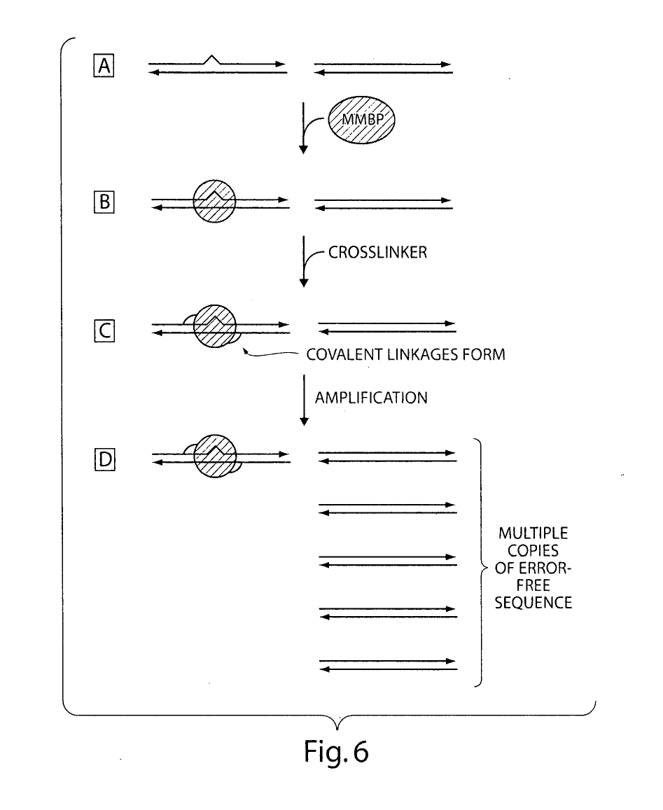

FIG. 6 illustrates an exemplary method for neutralizing sequence errors using mismatch recognition proteins. In this embodiment, the error-containing DNA sequence is not removed from the pool of DNA products. Rather, it becomes irreversibly complexed with a mismatch recognition protein by the action of a chemical crosslinking agent (for example, dimethyl suberimidate, DMS), or of another protein (such as MutL). The pool of DNA sequences is then amplified (such as by the polymerase chain reaction, PCR), but those containing errors are blocked from amplification, and quickly become outnumbered by the increasing error-free sequences. FIG. 6A illustrates an exemplary pool of DNA duplexes containing some duplexes with mismatches (left) and some which are error-free (right). A MMBP may be used to bind selectively to the DNA duplexes containing mismatches (FIG. 6B). The MMBP may be irreversibly attached at the site of the mismatch upon application of a crosslinking agent (FIG. 6C). In the presence of the covalently linked MMBP, amplification of the pool of DNA duplexes produces more copies of the error-free duplexes (FIG. 6D). The MMBP-mismatch DNA complex is unable to participate in amplification because the bound protein prevents the two strands of the duplex from dissociating. For long DNA duplexes, the regions outside the MMBP-bound site may be able to partially dissociate and participate in partial amplification of those (error-free) regions.

As increasingly longer sequences of DNA are generated, the fraction of sequences which are completely error-free diminishes. At some length, it becomes likely that there will be no molecule in the entire pool which contains a completely correct sequence. Thus, for the generation of extremely long segments of DNA, it can be useful to produce smaller units first which can be subjected to the above error control approaches. Then these segments can be combined to yield the larger full length product. However, if errors in these extremely long sequences can be corrected locally, without removing or neutralizing the entire long DNA duplex, then the more complex stepwise assembly process can be avoided.

Many biological DNA repair mechanisms rely on recognizing the site of a mutation (error) and then using a template strand (most likely error-free) to replace the incorrect sequence. In the de novo production of DNA sequences, this process is complicated by the difficulty of determining which strand contains the error and which should be used as the template. In this invention, the solutions to this problem rely on using the pool of other sequences in the mixture to provide the template for correction. These methods can be very robust: even if every strand of DNA contains one or more errors, as long as the majority of strands have the correct sequence at each position (expected because the positions of errors are generally not correlated between strands), there is a high likelihood that a given error will be replaced with the correct sequence. FIGS. 7, 8, 9, 10, 11, and 12 present exemplary procedures for performing this sort of local error correction.

FIG. 7 illustrates an exemplary method for carrying out strand-specific error correction. In replicating organisms, enzyme-mediated DNA methylation is often used to identify the template (parent) DNA strand. The newly synthesized (daughter) strand is at first unmethylated. When a mismatch is detected, the hemimethylated state of the duplex DNA is used to direct the mismatch repair system to make a correction to the daughter strand only. However, in the de novo synthesis of a pair of complementary DNA strands, both strands are unmethylated, and the repair system has no intrinsic basis for choosing which strand to correct. In this aspect of the invention, methylation and site-specific demethylation are employed to produce DNA strands that are selectively hemi-methylated. A methylase, such as the Dam methylase of E. coli, is used to uniformly methylate all potential target sites on each strand. The DNA strands are then dissociated, and allowed to re-anneal with new partner strands. A new protein is applied, a fusion of a mismatch binding protein (MMBP) with a demethylase. This fusion protein binds only to the mismatch, and the proximity of the demethylase removes methyl groups from either strand, but only near the site of the mismatch. A subsequent cycle of dissociation and annealing allows the (demethylated) error-containing strand to associate with a (methylated) strand which is error-free in this region of its sequence. (This should be true for the majority of the strands, since the locations of errors on complementary strands are not correlated.) The hemi-methylated DNA duplex now contains all the information needed to direct the repair of the error, employing the components of a DNA mismatch repair system, such as that of E. coli, which employs MutS, MutL, MutH, and DNA polymerase proteins for this purpose. The process can be repeated multiple times to ensure all errors are corrected.

FIG. 7A shows two DNA duplexes that are identical except for a single base error in the top left strand, giving rise to a mismatch. The strands of the right hand duplex are shown with thicker lines. Methylase (M) may then be used to uniformly methylates all possible sites on each DNA strand (FIG. 7B). The methylase is then removed, and a protein fusion is applied, containing both a mismatch binding protein (MMBP) and a demethylase (D) (FIG. 7C). The MMBP portion of the fusion protein binds to the site of the mismatch thus localizing the fusion protein to the site of the mismatch. The demethylase portion of the fusion protein may then act to specifically remove methyl groups from both strands in the vicinity of the mismatch (FIG. 7D). The MMBP-D protein fusion may then be removed, and the DNA duplexes may be allowed to dissociated and re-associate with new partner strands (FIG. 7E). The error-containing strand will most likely re-associate with a complementary strand which a) does not contain a complementary error at that site; and b) is methylated near the site of the mismatch. This new duplex now mimics the natural substrate for DNA mismatch repair systems. The components of a mismatch repair system (such as E. coli MutS, MutL, MutH, and DNA polymerase) may then be used to remove bases in the error-containing strand (including the error), and uses the opposing (error-free) strand as a template for synthesizing the replacement, leaving a corrected strand (FIG. 7F).

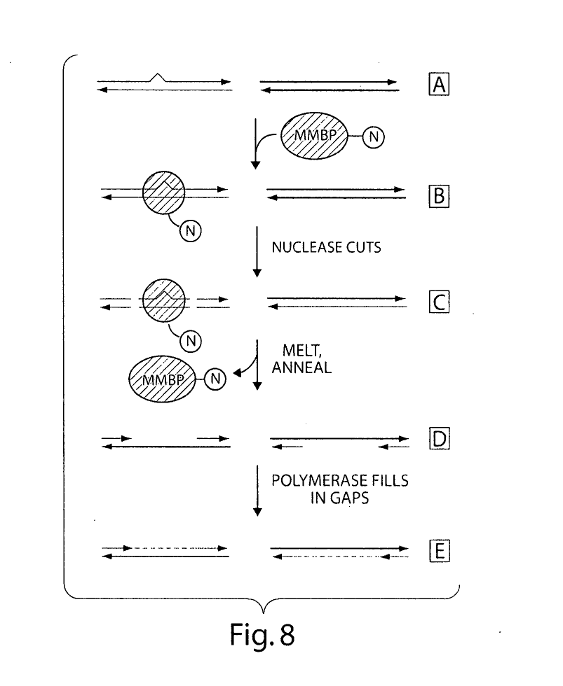

FIG. 8 illustrates an exemplary method for local removal of DNA on both strands at the site of a mismatch. Various proteins can be used to create a break in both DNA strands near an error. For example, an MMBP fusion to a non-specific nuclease (such as DNAseI) can direct the action of the nuclease (N) to the mismatch site, cleaving both strands. Once the break is generated, homologous recombination can be employed to use other strands (most of which will be error-free at this site) as template to replace the excised DNA. For example, the RecA protein can be used to facilitate single strand invasion, and early step in homologous recombination. Alternatively, a polymerase can be employed to allow broken strands to reassociate with new full-length partner strands, synthesizing new DNA to replace the error. For example, FIG. 8A shows two DNA duplexes that are identical except that one contains a single base error as in FIG. 7A. In one embodiment, a protein, such as a fusion of a MMBP with a nuclease (N), may be added and will bind at the site of the mismatch (FIG. 8B). Alternatively, a nuclease with specificity for single-stranded DNA can be employed, using elevated temperatures to favor local melting of the DNA duplex at the site of the mismatch. (In the absence of a mismatch, a perfect DNA duplex will be less likely to melt.) An endonuclease, such as that of the MMBP-N fusion, may be used to make double-stranded breaks near the site of the mismatch (FIG. 8C). The MMBP-N complex is then removed, along with the bound short region of DNA duplex around the mismatch (FIG. 8D). Melting and re-annealing of partner strands produces some duplexes with single-stranded gaps. A DNA polymerase may then be used to fill in the gaps, producing DNA duplexes without the original error (FIG. 8E).

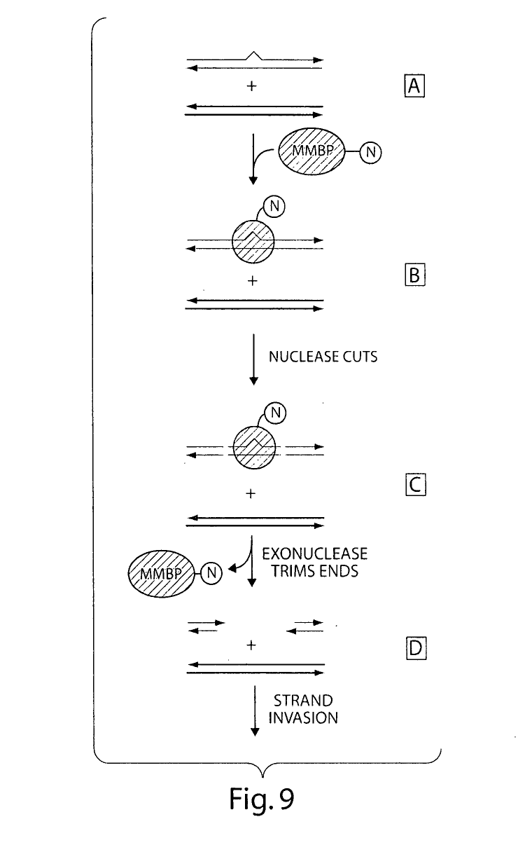

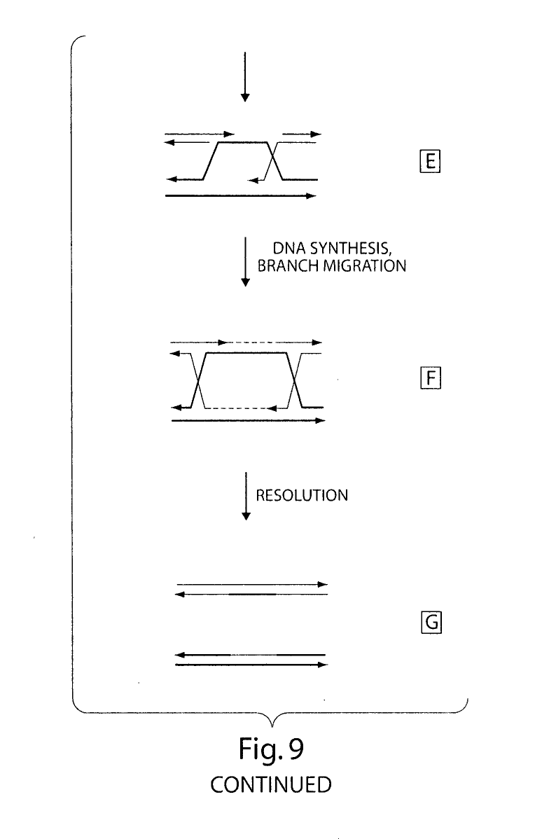

FIG. 9 illustrates a process similar to that of FIG. 8, however, in this embodiment, double-stranded gaps in DNA duplexes are repaired using the protein components of a recombination repair pathway. (Note that in this case no global melting and re-annealing of DNA strands is required, which can be preferable when dealing with especially large DNA molecules, such as genomic DNA.) For example, FIG. 9A shows two DNA duplexes (as in FIG. 8A), identical except that one contains a single base mismatch. As in FIG. 8B, a protein, such as a fusion of a MMBP with a nuclease (N), is added to bind at the site of the mismatch (FIG. 9B). As in FIG. 8C, an endonuclease, such as that of the MMBP-N fusion, may be used to make double-stranded breaks around the site of the mismatch (FIG. 9C). Protein components of a DNA repair pathway, such as the RecBCD complex, may then be employed to further digest the exposed ends of the double-stranded break, leaving 3′ overlaps (FIG. 9D). Subsequently, protein components of a DNA repair pathway, such as the RecA protein, are employed to facilitate single strand invasion of the intact DNA duplex, forming a Holliday junction (FIG. 9E). A DNA polymerase may then be used to synthesize new DNA, filling in the single-stranded gaps (FIG. 9F). Finally, protein components of a DNA repair pathway may be employed, such as the RuvC protein, to resolve the Holliday junction (FIG. 9G). The two resulting DNA duplexes do not contain the original error. Note that there can be more than one way to resolve such junctions, depending on migration of the branch points.

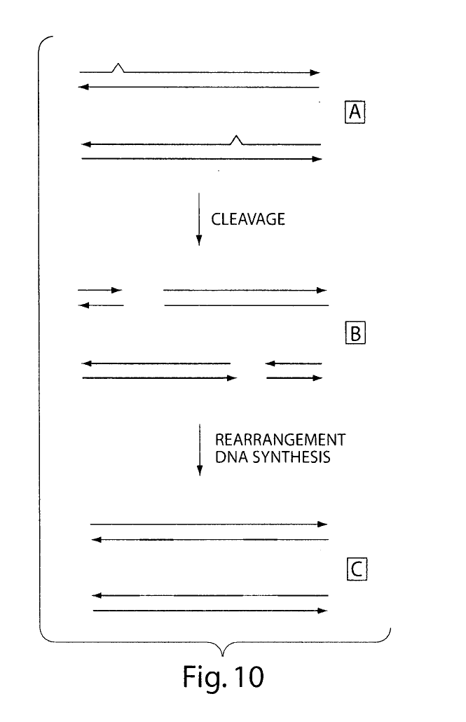

It is important to make clear that the methods described herein are capable of generating large error-free DNA sequences, even if none of the initial DNA products are error-free. FIG. 10 summarizes the effects of the methods of FIG. 8 (or equivalently, FIG. 9) applied to two DNA duplexes, each containing a single base (mismatch) error. For example, FIG. 10A illustrates two DNA duplexes, identical except for a single base mismatch in each, at different locations in the DNA sequence. Mismatch binding and localized nuclease activity are then used to generate double-stranded breaks which excise the errors (FIG. 10B). Recombination repair (as in FIG. 9) or melting and reassembly (as in FIG. 8) are employed to generate DNA duplexes where each excised error sequence has been replaced with newly synthesized sequence, each using the other DNA duplex as template (and unlikely to have an error in that same location) (FIG. 10C). Note that complete dissociation and re-annealing of the DNA duplexes is not necessary to generate the error-free products (if the methods shown in FIG. 9 are employed).

A simple way to reduce errors in long DNA molecules is to cleave both strands of the DNA backbone at multiple sites, such as with a site-specific endonuclease which generates short single stranded overhangs at the cleavage site. Of the resulting segments, some are expected to contain mismatches. These can be removed by the action and subsequent removal of a mismatch binding protein, as described in FIG. 5. The remaining pool of segments can be re-ligated into full length sequences. As with the approach of FIG. 9, this approach includes several advantages. 1) removal of an entire full length DNA duplex is not required to remove an error; 2) global dissociation and re-annealing of DNA duplexes is not necessary; 3) error-free DNA molecules can be constructed from a starting pool in which no one member is an error-free DNA molecule.

If the most common type of restriction endonuclease were employed for this approach, all DNA cleavage sites would result in identical overhangs. Thus the segments would associate and ligate in random order. However, use of a site-specific “outside cutter” (e.g., type IIS) endonuclease (such as HgaI, FokI, or BspMI) produces cleavage sites adjacent to (non-overlapping) the DNA recognition site. Thus each overhang would have sequence specific to that part of the DNA, distinct from that of the other sites. The re-association of these specifically complementary cohesive ends will then cause the segments to come together in the proper order. The cohesive ends generated can be up to five bases in length, allowing for up to 4⁵=1024 different combinations. Conceivably this many distinct restriction sites could be employed, though the need to avoid near matches between cohesive ends could lower this number.

The necessary restriction sites can be specifically included in the design of the sequence, or the random distribution of restriction sites within a desired sequence can be utilized (the recognition sequence of each endonuclease allows prediction of the typical distribution of fragments produced). Also, the target sequence can be analyzed for which choice of endonuclease produces the most ideal set of fragments.

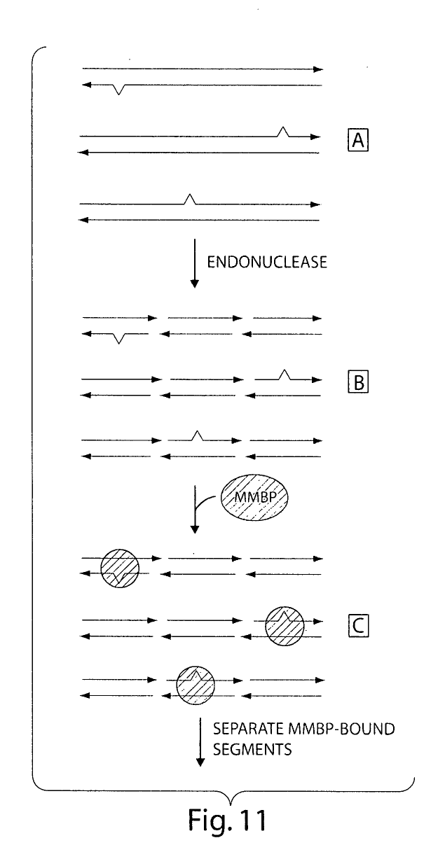

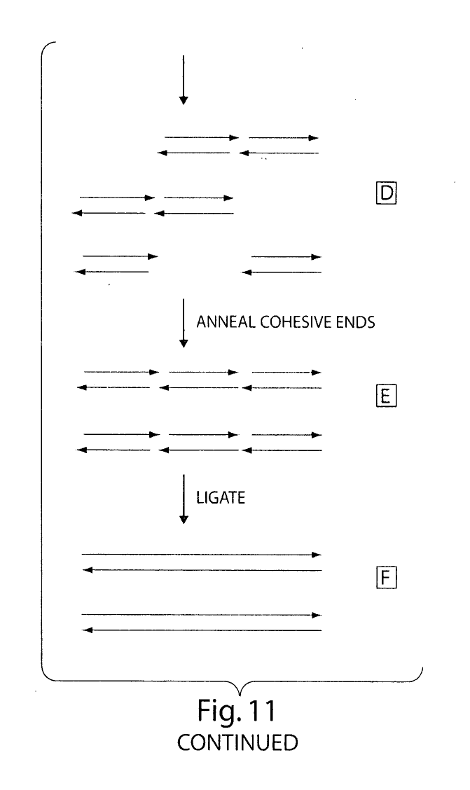

FIG. 11 shows an example of semi-selective removal of mismatch-containing segments. For example, FIG. 11A illustrates three DNA duplexes, each containing one error leading to a mismatch. The DNA is cut with a site-specific endonuclease, leaving double-stranded fragments with cohesive ends complementary to the adjacent segment (FIG. 11B). A MMBP is then applied, which binds to each fragment containing a mismatch (FIG. 11C). Fragments bound to MMBP are removed from the pool, as described in FIG. 5 (FIG. 11D). The cohesive ends of each fragment allow each DNA duplex to associate with the correct sequence-specific neighbor fragment (FIG. 11E). A ligase (such T4 DNA ligase) is employed to join the cohesive ends, producing full length DNA sequences (FIG. 11F). These DNA sequences can be error-free in spite of the fact that none of the original DNA duplexes was error-free. Incomplete ligation may leave some sequences which are less than full-length, which can be purified away on the basis of size.

The above approaches provide a major advantage over one of the conventional methods of removing errors, which employs sequencing first to find an error, and then relies on choosing specific error-free subsequences to “cut and paste” with endonuclease and ligase. In this embodiment, no sequencing or user choice is required in order to remove errors.

When complementary DNA strands are synthesized and allowed to anneal, both strands may contain errors, but the chance of errors occurring at the same base position in both sequences is extremely small, as discussed above. The above methods are useful for eliminating the majority case of uncorrelated errors which can be detected as DNA mismatches. In the rare case of complementary errors at identical positions on both strands (undetectable by the mismatch binding proteins), a subsequent cycle of duplex dissociation and random re-annealing with a different complementary strand (with a different distribution of error positions) remedies the problem. But in some applications it is desirable to not melt and re-anneal the DNA duplexes, such as in the case of genomic-length DNA strands. In such an embodiment, correlated errors may be removed using a different method. For example, though the initial population of correlated errors is expected to be low, amplification or other replication of the DNA sequences in a pool will ensure that each error is copied to produce a perfectly complementary strand which contains the complementary error. According to the invention that this approach does not require global dissociation and re-annealing of the DNA strands. Essentially, various forms of DNA damage and recombination are employed to allow single-stranded portions of the long DNA duplex to re-assort into different duplexes.

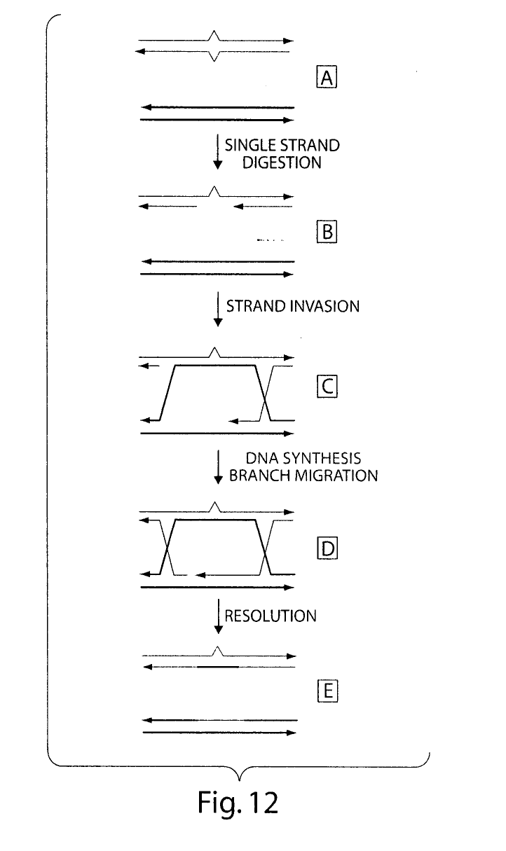

FIG. 12 shows a procedure for reducing correlated errors in synthesized DNA. FIG. 12A shows two DNA duplexes identical except for a single error in one strand. Non-specific nucleases may be used to generate short single-stranded gaps in random locations in the DNA duplexes in the pool (FIG. 12B). Shown here is the result of one of these gaps generated at the site of one of the correlated locations. Recombination-specific proteins such as RecA and RuvB are employed to mediate the formation of a four-stranded Holliday junction (FIG. 12C). DNA polymerase is employed to fill in the gap shown in the lower portion of the complex (FIG. 12D). Action of other recombination and/or repair proteins such as RuvC is employed to cleave the Holliday junction, resulting in two new DNA duplexes, containing some sequences which are hybrids of their progenitors (FIG. 12E). In the example shown, one of the error-containing regions has been eliminated. However, since the cutting, rearrangement, and replacement of strands employed in this method is intended to be random, it is expected that the total number of errors in the sequence will actually not change, simply that errors will be reassorted to different strands. Thus, pairs of errors correlated in one duplex will be reshuffled into separate duplexes, each with a single error. This random reassortment of strands will yield new duplexes containing mismatches which can be repaired using the mismatch repair proteins detailed above. Unique to this embodiment of the invention is the use of recombination to separate the correlated errors into different DNA duplexes.

This process makes possible the direct fabrication of DNA of any desired sequence. No longer do expression vectors have to be constructed from component parts by techniques of in vitro recombinant DNA. Instead, any desired DNA construct can be directly synthesized in total by direct synthesis in segments followed by spontaneous assembly into the completed molecule. The constructed DNA molecule does not have to be one that previously existed, it can be a totally novel construct to suit a particular purpose. It now becomes possible for one of skill in the art to design a desired DNA sequence or vector entirely in the computer, and then to directly synthesize the DNA vector artificially in a single series of operations.

It is envisioned that the process of direct DNA synthesis envisioned here will begin with a desired target DNA sequence, in the form of a computer file representing the target sequence that the user wants to build. A computer software program is used to determine the optimal way to subdivide the desired DNA construct into smaller DNA that can be used to build the larger target sequence. The software would be optimized for this purpose. For example, the target DNA construct should be subdivided into segments in such a manner so that the hybridizing half of each segment will hybridize well to a corresponding half segment, and not to any other half segment. If needed, changes to the sequence not affecting the ultimate functionality of the DNA may be required in some instances to ensure unique segments. This sort of optimization is preferable done by computer systems designed for this purpose.

After the DNA segments are constructed on the substrate of the microarray, the DNA segments must be separated from the microarray substrate. This can be done by any of a number of techniques, depending on the technique used to attach the DNA segments to the substrate in the first place. Described below is one technique based on base labile chemistry, adapted from techniques used to fabricate oligonucleotides on glass particles, but this is only one example among several possibilities. In essence, all that is required is that the attachment of the DNA segments to the substrate be cleaved by a technique that does not destroy the DNA molecules themselves.

This process may or may not make enough directly synthesized DNA as needed for a particular application. It is envisioned that more copies of the synthesized DNA can be made by any of the several ways in which other DNA constructs are cloned or replicated in quantity. An origin of replication can be built into circular DNA which would permit the rapid amplification of copies of the constructed DNA in a bacterial host. Linear DNA can be constructed with defined DNA primers at each end which can then be used to amplify many copies of the DNA construct by the PCR process.

### Example 3

### Synthetic Genomes

The hierarchical assembly methods described herein may be used, for example, to construct large polynucleotide constructs that may not be constructed ex vivo (or at least not easily constructed ex vivo) due to shearing and other difficulties in manipulating large nucleotide constructs. The methods may also be used for making genome wide nucleotide alterations in a cell. For example, producing a genome having a plurality of sequence alterations scattered throughout the genome may be achieved using the hierarchical assembly methods described herein. Such methods may be used to produce a genome having a plurality of specific and predetermined nucleotide substitutions, for example, at least about 50, 100, 200, 500, 750, 1000, 2000, 5000, 10000, or more, specific nucleotide alterations at different and predetermined locations throughout the genome. In an exemplary embodiment, the hierarchical assembly methods described herein may be used to produce a cell in which one or more codons have been replaced. Such a cell may have a different pattern of codon usage as compared to a wild-type cell. In certain embodiments, the cell does not use one or more codons (e.g., one or two stop codons) for the expression of endogenous genes and these codons are available for use in a nucleic acid template encoding an artificial polypeptide.

A transposable genetic element is a genetic unit, such as a transposon, that can insert into, exit from, or relocate within a genome, chromosome, or plasmid. A transposon is a region of a genome that may be flanked by inverted repeats, direct repeats, or no significant repeats. A copy of the transposon can be inserted at a different location in the genetic material of an organism. Typically a transposon includes a gene encoding a transposase which is a protein that catalyzes the transposition of the genetic element, including the DNA encoding the transposase itself. A transposase may induce integration at random locations in some or all species in which it is operative, or it may insert the element at a specific site, and may behave differently in different species (see, e.g., Osborn et al, Plasmid 48: 202-212 (2002)). Transposable genetic elements are active in many microorganisms, and serve to protect the species from insertions, to relieve metabolic stresses, and to drive evolutionary change. Microbial expression vehicles, which are laboriously engineered to maximize expression of a gene or synthesis of a small molecule or polymer through a metabolic pathway, often lose their engineered phenotype through transposition of genetic elements. Thus, in a matter of a few generations, clones which have experienced a transposition event that inactivates overexpression, or otherwise serves to give the cell a metabolic advantage, or relieve metabolic stress, can swamp the culture.

In accordance with one aspect of the invention, a transposon knock down cell may be produced as an expression vehicle having improved phenotypic stability, or for other purposes. This is accomplished by intentionally mutating the DNA encoding the ORFs (or control elements) of transposase enzymes, preferably all copies of all transposase enzymes in the genome of a cell, so that they are inoperative. This inactivates DNA segment jumping within a cell and among cells of the same clone, and reduces the frequency and speed of spontaneous reversion of carefully engineered cells to wild type characteristics. This may be done together with other efforts to modify the genome of an organism as discussed herein, or alone by genome-wide point mutations. Recombination into the genome of specially designed synthetic DNA is ideal for this purpose. In certain embodiments, the point mutations may be in a region that effects expression of the transposase, for example, in the coding regions, or a region that controls expression of the transposase. Alternatively, the point mutations may be made in regions flanking the coding region of the transposase that facilitate copying, excision, or insertion of the transposon element. For example, in case where the transposon is flanked by inverted repeats, the point mutations may be made in the inverted repeats. Various combinations of point mutations in the transposase coding region, transposase transcriptional and/or translational control sequences, and/or flanking regions mare also contemplated. In certain embodiments, a single transposon may be inactivated by introduction of one or more point mutations, including, 1, 2, 3, 4, 5, 6, 7, 8, 9, 10, or more, point mutations in a single transposon. In an exemplary embodiment, at least one point mutation that terminates translation of a transposase (e.g., a stop codon) is introduced into the DNA sequence such that translation of the protein is terminated near the N-terminus, e.g., within 2, 5, 10, 15, 20, 25, 30, or 50 amino acids of the N-terminus of the transposase. In yet another embodiment, two or more stop codons are introduced toward the 5′ end of the transposase sequence. In certain embodiments, the genome size and/or spacing is not changed upon introduction of the point mutations. In an exemplary embodiment, the point mutations modify one or more bases to different bases but do not delete any bases from the genome.

In yet other embodiments, mutations in conjugation, mating, and transformation apparatus may be introduced. When combined with other genome modifications as described herein, significantly higher safety may be achieved.

The methods provided herein also permit construction of organisms having an altered genetic code. For example, the ability to make genome wide modifications permits the construction of organisms that utilize a different codon complement and/or a different tRNA complement as compared to a wild-type cell. Such organisms may be genetically isolated from wild-type organism because they cannot donate sequences that are properly expressed in wild-type cells and/or they cannot properly express sequences received from a wild-type organism. Additionally, genome wide modifications permit production of proteins that contain unnatural amino acids, e.g., by using altered tRNA molecules that are charged with an unnatural amino acid and will insert the amino acid in response to a desired codon. Additionally, it is possible to construct organisms that have an altered organelle genome (e.g., mitochondrial genome or chloroplast genome). It may be possible to genetically alter chromosomal DNA and/or extrachromosomal DNA in a given cell.

### Example 4

### Homologous Recombination/Site-Specific Recombination

In certain embodiments, large polynucleotide constructs such as synthetic genomes may be produced by replacing portions of the genome with corresponding portions containing the desired sequence modifications. For example, this may be achieved by homologous recombination of long DNA molecules prepared as described above and containing the desired sequence substitutions. Alternatively, site-specific recombination using one or more integrases may be used to create a synthetic genome.

Homologous recombination (or general recombination) is defined as the exchange of homologous segments anywhere along a length of two DNA molecules. An essential feature of general recombination is that the enzymes responsible for the recombination event can presumably use any pair of homologous sequences as substrates, although some types of sequence may be favored over others.

Alternatively, in site-specific recombination, exchange occurs at a specific site, as in the integration of phage λ into the E. coli chromosome and the excision of λ DNA from the E. coli chromosome. Site-specific recombination involves specific (e.g., inverted repeat, non-repetitive, etc.) sequences; e.g. the Cre-loxP and FLP-FRT systems. Within these sequences there is only a short stretch of homology necessary for the recombination event, but not sufficient for it. The enzymes involved in this event generally cannot recombine other pairs of homologous (or nonhomologous) sequences, but act specifically.

Although both site-specific recombination and homologous recombination are useful mechanisms for genetic engineering of DNA sequences, targeted homologous recombination provides a basis for targeting and altering essentially any desired sequence in a duplex DNA molecule, such as targeting a DNA sequence in a chromosome for replacement by another sequence. Site-specific recombination has been proposed as one method to integrate transfected DNA at chromosomal locations having specific recognition sites (O'Gorman et al. (1991) Science 251: 1351; Onouchi et al. (1991) Nucleic Acids Res. 19: 6373). Unfortunately, since this approach requires the presence of specific target sequences and recombinases, its utility for targeting recombination events at any particular chromosomal location is severely limited in comparison to targeted general recombination. Mitigating this requirement is the availability of designed site-specific recombination initiators (see e.g., Urnov F D, et al. Nature 2005 (epublication on Apr. 3, 2005) entitled Highly efficient endogenous human gene correction using designed zinc-finger nucleases).

A primary step in homologous recombination is DNA strand exchange, which involves a pairing of a DNA duplex with at least one DNA strand containing a complementary sequence to form an intermediate recombination structure containing heteroduplex DNA (see, Radding, C. M. (1982) Ann. Rev. Genet. 16: 405; U.S. Pat. No. 4,888,274). The heteroduplex DNA may take several forms, including a three DNA strand containing triplex form wherein a single complementary strand invades the DNA duplex (Hsieh et al. (1990) Genes and Development 4: 1951; Rao et al., (1991) PNAS 88:2984)) and, when two complementary DNA strands pair with a DNA duplex, a classical Holliday recombination joint or chi structure (Holliday, R. (1964) Genet. Res. 5: 282) may form, or a double-D loop. Once formed, a heteroduplex structure may be resolved by strand breakage and exchange, so that all or a portion of an invading DNA strand is spliced into a recipient DNA duplex, adding or replacing a segment of the recipient DNA duplex. Alternatively, a heteroduplex structure may result in gene conversion, wherein a sequence of an invading strand is transferred to a recipient DNA duplex by repair of mismatched bases using the invading strand as a template (Genes, 3rd Ed. (1987) Lewin, B., John Wiley, New York, N.Y.; Lopez et al. (1987) Nucleic Acids Res. 15: 5643). Whether by the mechanism of breakage and rejoining or by the mechanism(s) of gene conversion, formation of heteroduplex DNA at homologously paired joints can serve to transfer genetic sequence information from one DNA molecule to another.

The ability of homologous recombination (gene conversion and classical strand breakage/rejoining) to transfer genetic sequence information between DNA molecules makes targeted homologous recombination a powerful method in genetic engineering and gene manipulation.

The ability of cells to incorporate exogenous genetic material into genes residing on chromosomes has demonstrated that some cells (including yeast, mammals and humans) have the general enzymatic machinery for carrying out homologous recombination required between resident and introduced sequences. These targeted recombination events can be used to correct mutations at known sites, replace genes or gene segments with defective ones, or introduce foreign genes into cells. The efficiency of such gene targeting techniques is related to several parameters: the efficiency of DNA delivery into cells, the type of DNA packaging (if any) and the size and conformation of the incoming DNA, the length and position of regions homologous to the target site (all these parameters also likely affect the ability of the incoming homologous DNA sequences to survive intracellular nuclease attack), the efficiency of hybridization and recombination at particular chromosomal sites and whether recombinant events are homologous or nonhomologous.

Unfortunately, exogenous sequences transferred into eukaryotic cells undergo homologous recombination with homologous endogenous sequences only at very low frequencies, and are so inefficiently recombined that large numbers of cells must be transfected, selected, and screened in order to generate a desired correctly targeted homologous recombinant (Kucherlapati et al. (1984) Proc. Natl. Acad. Sci. (U.S.A.) 81: 3153; Smithies, O. (1985) Nature 317: 230; Song et al. (1987) Proc. Natl. Acad. Sci. (U.S.A.) 84: 6820; Doetschman et al. (1987) Nature 330: 576; Kim and Smithies (1988) Nucleic Acids Res. 16: 8887; Doetschman et al. (1988) op.cit.; Koller and Smithies (1989) op.cit.; Shesely et al. (1991) Proc. Natl. Acad. Sci. (U.S.A.) 88: 4294; Kim et al. (1991) Gene 103: 227, which are incorporated herein by reference).

Koller et al. (1991) Proc. Natl. Acad. Sci. (U.S.A.), 88: 10730 and Snouwaert et al. (1992) Science 257: 1083, have described targeting of the mouse cystic fibrosis transmembrane regulator (CFTR) gene for the purpose of inactivating, rather than correcting, a murine CFTR allele. Koller et al. employed a large (7.8 kb) homology region in the targeting construct, but nonetheless reported a low frequency for correct targeting (only 1 of 2500 G418-resistant cells were correctly targeted). Thus, even targeting constructs having long homology regions are inefficiently targeted.

Several proteins or purified extracts having the property of promoting homologous recombination (i.e., recombinase activity) have been identified in prokaryotes and eukaryotes (Cox and Lehman (1987) Ann. Rev. Biochem. 56: 229; Radding, C. M. (1982) op.cit.; Madiraju et al. (1988) Proc. Natl. Acad. Sci. (U.S.A.) 85: 6592; McCarthy et al. (1988) Proc. Natl. Acad. Sci. (U.S.A.) 85: 5854; Lopez et al. (1987) op.cit., which are incorporated herein by reference). These general recombinases presumably promote one or more steps in the formation of homologously-paired intermediates, strand-exchange, gene conversion, and/or other steps in the process of homologous recombination.

The frequency of homologous recombination in prokaryotes is significantly enhanced by the presence of recombinase activities. Several purified proteins catalyze homologous pairing and/or strand exchange in vitro, including: E. coli recA protein, the T4 uvsX protein, the rec 1 protein from Ustilago maydis, and Rad51 protein from S. cervisiae (Sung et al., Science 265:1241 (1994)) and human cells (Baumann et al., Cell 87:757 (1996)). Recombinases, like the recA protein of E. coli are proteins which promote strand pairing and exchange. The most studied recombinase to date has been the recA recombinase of E. coli, which is involved in homology search and strand exchange reactions (see, Cox and Lehman (1987) op.cit.). RecA is required for induction of the SOS repair response, DNA repair, and efficient genetic recombination in E. coli. RecA can catalyze homologous pairing of a linear duplex DNA and a homologous single strand DNA in vitro. In contrast to site-specific recombinases, proteins like recA which are involved in general recombination recognize and promote pairing of DNA structures on the basis of shared homology, as has been shown by several in vitro experiments (Hsieh and Camerini-Otero (1989) J. Biol. Chem. 264: 5089; Howard-Flanders et al. (1984) Nature 309: 215; Stasiak et al. (1984) Cold Spring Harbor Symp. Quant. Biol. 49: 561; Register et al. (1987) J. Biol. Chem. 262: 12812). Several investigators have used recA protein in vitro to promote homologously paired triplex DNA (Cheng et al. (1988) J. Biol. Chem. 263: 15110; Ferrin and Camerini-Otero (1991) Science 354: 1494; Ramdas et al. (1989) J. Biol. Chem. 264: 11395; Strobel et al. (1991) Science 254: 1639; Hsieh et al. (1990) op.cit.; Rigas et al. (1986) Proc. Natl. Acad. Sci. (U.S.A.) 83: 9591; and Camerini-Otero et al. U.S. Pat. No. 7,611,268 (available from Derwent), which are incorporated herein by reference).

Common mechanisms for inducing recombination include, but are not limited to the use of strains comprising mutations such as those involved in mismatch repair. e.g. mutations in mutS, mutT, mutL and muth; exposure to U.V. light; Chemical mutagenesis, e.g. use of inhibitors of MMR, DNA damage inducible genes, or SOS inducers; overproduction/underproduction/mutation of any component of the homologous recombination complex/pathway, e.g. RecA, ssb, etc.; overproduction/underproduction/mutation of genes involved in DNA synthesis/homeostasis; overproduction/underproduction/mutation of recombination-stimulating genes from bacteria, phage (e.g. Lambda Red function), or other organisms; addition of chi sites into/flanking the donor DNA fragments; coating the DNA fragments with RecA/ssb and the like.

In certain embodiments, the host cell may naturally be capable of carrying out homologous recombination. Recombination generally occurs through the activity of one or more polypeptides which form a “recombination system.” In some embodiments the host cell may contain an endogenous recombination system. In other embodiments, the host cell may contain an endogenous recombination system that may be enhanced by one or more exogenous factors that facilitate recombination in the host cell. For example, the host cell may be engineered to express a polypeptide involved in recombination or a recombination facilitating factor may be mixed with a targeting polynucleotide prior to its introduction into the host cell. In still other embodiments, the host cell may be been engineered to comprise a homologous recombination system that is not endogenous to the cell.

In an exemplary embodiment, a host cell comprises a recombination system having one or more polypeptides encoded by the genes selected from the group consisting of the exo, bet and gam genes from phage λ. The gam gene (also referred to as gamma or γ) encodes a protein which inhibits the RecBCD nuclease from degrading linear DNA while the exo and bet (also referred to as beta or β) genes encode proteins involved in homologous recombination. In one embodiment, the homologous recombination system is the phage λ recombinase system comprising the exo, bet and gam genes of phage λ. Still other suitable recombination systems will be known to one of skill in the art.

The “stuffer” fragment of lambda 1059 carries the lambda exo, beta, gamma under the control of the leftward promoter (pL). These genes confer an Spi+phenotype, i.e., the phage is able to grow on recA⁻ strains but is unable to grow on strains that are lysogenic for bacteriophage P2. Since pL is also located on the “stuffer” fragment, the expression of the Spi+phenotype is not affected by the orientation of the “stuffer” between the left and right arms of the vector.

Wild-type members of the Enterobacteriaceae (e.g., Escherichia coli) are typically resistant to genetic exchange following transformation of linear DNA molecules. This is due, at least in part, to the Exonuclease V (Exo V) activity of the RecBCD holoenzyme which rapidly degrades linear DNA molecules following transformation. Production of ExoV has been traced to the recD gene, which encodes the D subunit of the holoenzyme. The RecBCD holoenzyme plays an important role in initiation of RecA-dependent homologous recombination. Upon recognizing a dsDNA end, the RecBCD enzyme unwinds and degrades the DNA asymmetrically in a 5′ to 3′ direction until it encounters a chi (or “X”)-site (consensus 5′-GCTGGTGG-3′) which attenuates the nuclease activity. This results in the generation of a ssDNA terminating near the c site with a 3′-ssDNA tail that is preferred for RecA loading and subsequent invasion of dsDNA for homologous recombination. Accordingly, preprocessing of transforming fragments with a 5′ to 3′ specific ssDNA Exonuclease, such as Lambda (λ) exonuclease (available, e.g., from Boeringer Mannheim) prior to transformation may serve to stimulate homologous recombination in recD⁻ strains by providing ssDNA invasive end for RecA loading and subsequent strand invasion.

The addition sequences encoding chi-sites (consensus 5′-GCTGGTGG-3′) to DNA fragments can serve to both attenuate Exonuclease V activity and stimulate homologous recombination, thereby obviating the need for a recD mutation (see also, Kowalczykowski, et al. (1994) “Biochemistry of a homologous recombination in Escherichia coli,”

Microbiol. Rev. 58:401-465 and Jessen, et al. (1998) “Modification of bacterial artificial chromosomes through Chi-stimulated homologous recombination and its application in zebra fish transgenesis.” Proc. Natl. Acad. Sci. 95:5121-5126).

In certain embodiments, chi-sites may be included in the targeting polynucleotides described herein. The use of recombination-stimulatory sequences such as chi is a generally useful approach for increasing the efficiency of homologous recombination in a wide variety of cell types.

Methods to inhibit or mutate analogs of Exo V or other nucleases (such as, Exonucleases I (endA1), III (nth), IV (nfo), VII, and VIII of E. coli) is similarly useful. Inhibition or elimination of such nucleases, or modification of ends of transforming DNA fragments to render them resistant to exonuclease activity has applications in facilitating homologous recombination in a broad range of cell types.

In certain embodiments, a homologous recombination system may comprise one or more endogenous and/or exogenous recombinase proteins. Recombinases are proteins that may provide a measurable increase in the recombination frequency and/or localization frequency between a targeting polynucleotide and a desired target sequence. The most common recombinase is a family of RecA-like recombination proteins all having essentially all or most of the same functions, particularly: (i) the recombinase protein's ability to properly bind to and position targeting polynucleotides on their homologous targets and (ii) the ability of recombinase protein/targeting polynucleotide complexes to efficiently find and bind to complementary endogenous sequences. The best characterized recA protein is from E. coli, in addition to the wild-type protein a number of mutant recA-like proteins have been identified (e.g., recA803). Further, many organisms have recA-like recombinases with strand-transfer activities (e.g., Fugisawa et al., (1985) Nucl. Acids Res. 13: 7473; Hsieh et al., (1986) Cell 44: 885; Hsieh et al., (1989) J. Biol. Chem. 264: 5089; Fishel et al., (1988) Proc. Natl. Acad. Sci. USA 85: 3683; Cassuto et al., (1987) Mol. Gen. Genet. 208: 10; Ganea et al., (1987) Mol. Cell. Biol. 7: 3124; Moore et al., (1990) J. Biol. Chem. 19: 11108; Keene et al., (1984) Nucl. Acids Res. 12: 3057; Kimiec, (1984) Cold Spring Harbor Symp. 48:675; Kimeic, (1986) Cell 44: 545; Kolodner et al., (1987) Proc. Natl. Acad. Sci. USA 84:5560; Sugino et al., (1985) Proc. Natl. Acad, Sci. USA 85: 3683; Halbrook et al., (1989) J. Biol. Chem. 264: 21403; Eisen et al., (1988) Proc. Natl. Acad. Sci. USA 85: 7481; McCarthy et al., (1988) Proc. Natl. Acad. Sci. USA 85: 5854; Lowenhaupt et al., (1989) J. Biol. Chem. 264: 20568, which are incorporated herein by reference. Examples of such recombinase proteins include, for example but not limitation: recA, recA803, uvsX, and other recA mutants and recA-like recombinases (Roca, A. I. (1990) Crit. Rev. Biochem. Molec. Biol. 25: 415), sepI (Kolodner et al. (1987) Proc. Natl. Acad. Sci. (U.S.A.) 84: 5560; Tishkoff et al. Molec. Cell. Biol. 11: 2593), RuvC (Dunderdale et al. (1991) Nature 354: 506), DST2, KEM1, XRN1 (Dykstra et al. (1991) Molec. Cell. Biol. 11: 2583), STP-alpha/DST1 (Clark et al. (1991) Molec. Cell. Biol. 11: 2576), HPP-1 (Moore et al. (1991) Proc. Natl. Acad. Sci. (U.S.A.) 88: 9067), other eukaryotic recombinases (Bishop et al. (1992) Cell 69: 439; Shinohara et al. (1992) Cell 69: 457); incorporated herein by reference. RecA may be purified from E. coli strains, such as E. coli strains JC12772 and JC15369 (available from A. J. Clark and M. Madiraju, University of California-Berkeley). These strains contain the recA coding sequences on a “runaway” replicating plasmid vector present at a high copy numbers per cell. The recA803 protein is a high-activity mutant of wild-type recA. The art teaches several examples of recombinase proteins, for example, from Drosophila, yeast, plant, human, and non-human mammalian cells, including proteins with biological properties similar to recA (i.e., recA-like recombinases).

In certain embodiments, recombinase protein(s) (prokaryotic or eukaryotic) may be exogenously administered to a host cell. Such administration is typically done by microinjection, although electroporation, lipofection, and other transfection methods known in the art may also be used. Alternatively, recombinase proteins may be produced in vivo from a heterologous expression cassette in a transfected cell or transgenic cell, such as a transgenic totipotent embryonal stem cell (e.g., a murine ES cell such as AB-1) used to generate a transgenic non-human animal line or a pluripotent hematopoietic stem cell for reconstituting all or part of the hematopoietic stem cell population of an individual. In exemplary embodiments, a heterologous expression cassette may include a modulatable promoter, such as an ecdysone-inducible promoter-enhancer combination, an estrogen-induced promoter-enhancer combination, a CMV promoter-enhancer, an insulin gene promoter, or other cell-type specific, developmental stage-specific, hormone-inducible, or other modulatable promoter construct so that expression of at least one species of recombinase protein from the cassette can by modulated for transiently producing recombinase(s) in vivo simultaneous or contemporaneous with introduction of a targeting polynucleotide into the cell. When a hormone-inducible promoter-enhancer combination is used, the cell must have the required hormone receptor present, either naturally or as a consequence of expression a co-transfected expression vector encoding such receptor.

For making transgenic non-human animals (which include homologously targeted non-human animals) embryonal stem cells (ES cells) are preferred. Murine ES cells, such as AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 87: 27-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal).

The pluripotence of any given ES cell line can vary with time in culture and the care with which it has been handled. The only definitive assay for pluripotence is to determine whether the specific population of ES cells to be used for targeting can give rise to chimeras capable of germ line transmission of the ES genome. For this reason, prior to gene targeting, a portion of the parental population of AB-1 cells is injected into C57B1/6J blastocysts to ascertain whether the cells are capable of generating chimeric mice with extensive ES cell contribution and whether the majority of these chimeras can transmit the ES genome to progeny.

In another embodiment, site-specific recombination using one or more site-specific recombinases may be used to create a large polynucleotide construct such as a synthetic genome. A site-specific recombinase refers to a type of recombinase which typically has at least the following four activities (or combinations thereof): (1) recognition of one or two specific nucleic acid sequences; (2) cleavage of said sequence or sequences; (3) topoisomerase activity involved in strand exchange; and (4) ligase activity to reseal the cleaved strands of nucleic acid. See Sauer, B., Current Opinions in Biotechnology 5:521-527 (1994). The strand exchange mechanism involves the cleavage and rejoining of specific DNA sequences in the absence of DNA synthesis (Landy, A. (1989) Ann. Rev. Biochem. 58:913-949). Examples of site-specific recombinases include the integrase family of proteins and the tyrosine recombinase family of proteins (See e.g., Esposito and Scocca, Nucleic Acids Research 25: 3605-3614 (1997); Nunes-Duby et al., Nucleic acids Research 26: 391-406 (1998); and U.S. Patent Application Publication Nos. 2003/0124555 and 2003/0077804). In certain embodiments, a site-specific recombinase may naturally be present in the cell which is to be used for assembly, for example, when the cell is a bacterial cell or a yeast cell. In such an embodiment, a nucleic acid molecule may be introduced into the cell and the endogenous site-specific recombinase will catalyze integration of the nucleic acid into the appropriate location in the genome. Alternatively, when the cell to be used for assembly does not naturally contain a site-specific recombinase, a nucleic acid encoding the recombinase may introduced into the cell along with (either simultaneously or sequentially) the nucleic acid molecule to be inserted into the genome. The coding sequence for the recombinase may be integrated into the genome of the host cell, or may be maintained on a plasmid either stably or transiently.

In an exemplary embodiment, the site-specific recombinase is a tyrosine recombinase that targets insertion of a nucleic acid to a tRNA gene. Such tyrosine recombinases will be useful for modifying the tRNA complement in a synthetic genome, for example, by deleting or inactivating a wild-type tRNA, by modifying an endogenous tRNA (e.g., by modifying the sequence of the tRNA gene, such as, for example, the sequence of the anticodon loop, D loop, variable loop, TψC loop, acceptor, etc.), and/or by replacing a wild-type tRNA gene with a modified tRNA gene. Examples of tyrosine recombinases that target nucleic acid insertion to a tRNA gene include, for example, HP1 (Cell 89: 227-37 (1997)), L5 (J. Bact. 181: 454-61 (1999)), DLP12 (J. Bacteriol. 171: 6197-205 (1989)), P4 (J. Mol. Biol. 196: 487-96 (1987)), P22 (J. Biol. Chem. 260: 4468-77 (1985)), P2 (J. Bacteriol. 175: 1239-49 (1993)), P186 (J. Mol. Biol. 191: 199-209 (1986)), phiR73 (J. Bacteriol. 173: 4171-81)), RP3 (Nucl. Acids Res. 23: 58-63 (1995)), phiCTX (Mol. Gen. Genet. 246:72-79 (1995)), MV4 (J. Bacteriol. 179: 1837-45 (1997)), SSV1 (Mol. Gen. Genet. 237: 334-42 (1993)), T12 (Mol. Microb. 23: 719-28 (1997)), A2 (Virology 250: 185-93 (1998)), PPu orf (J. Bacteriol. 180: 5505-14 (1998)), phi10MC (FEMS Microb. Let. 147: 279-85 (1997)), VWB (Microbiology 144: 3351-58 (1998)), and YPe of (Mol. Microb. 31(1): 291-303 (1999)).

### Example 5

### Exemplary Organisms

The methods described herein may be used to produce synthetic genomes for a variety of cells, including eukaryotic, prokaryotic, diploid, or haploid organisms. Host organisms with synthetic genomes may be used to express any of the polypeptides or proteins described herein. The resulting organisms having synthetic genomes may be single cell organisms (e.g., bacteria, e.g., E. coli, e.g., B. subtilis, e.g., Mycobacterium spp., e.g., M. tuberculosis) or may be derived from multicellular organisms (transgenic organisms, such as insects (e.g., Drosophila), worms (e.g., Caenorhabditis spp, e.g., C. elegans) and higher animals (e.g., transgenic mammals such as mice, rats, rabbits, hamsters, etc.). In certain embodiments, a cell having a synthetic genome is a naturally diploid cell, preferably yeast cells (e.g., Saccharomyces spp. (e.g., S. cerevisiae), Candida spp. (e.g., C. albicans)) or mammalian cells (e.g., mouse, monkey, or human). These cells may be used as host cells for artificial polypeptide expression as described herein. A host cell may be used to express a polypeptides or protein from a different species of organism or an entirely synthetic polypeptide.

It should be appreciated that a host cell is preferably a safe cell that does not produce any toxins or is non-infectious (i.e., does not cause any disease). However, in certain embodiments, a toxic, infectious, or other type of cell may be used as a host if that cell possesses certain characteristics that are necessary for expressing a polypeptide of interest (e.g., certain transcription or translational properties or certain post-translational modification properties such as unique glycosylation, etc.) Also, certain cell types or species may have a very low usage of a particular codon that is to be used for unnatural amino acid incorporation. These cells or species may be useful to generate appropriate host cells since they would require less genetic modification to remove the chosen threshold number of selected codons from their genome.

Any means for the introduction of polynucleotides into eukaryotic or prokaryotic cells may be used in accordance with the compositions and methods described herein. Suitable methods include, for example, direct needle microinjection, transfection, electroporation, retroviruses, adenoviruses, adeno-associated viruses; Herpes viruses, and other viral packaging and delivery systems, polyamidoamine dendrimers, liposomes, and more recently techniques using DNA-coated microprojectiles delivered with a gene gun (called a biolistics device), or narrow-beam lasers (laser-poration). In one embodiment, nucleic acid constructs may be delivered in a complex with a colloidal dispersion system. A colloidal system includes macromolecule complexes, nanocapsules, microspheres, beads, and lipid-based systems including oil-in-water emulsions, micelles, mixed micelles, and liposomes. An exemplary colloidal system of this invention is a lipid-complexed or liposome-formulated DNA. See, e.g., Canonico et al, Am J Respir Cell Mol Biol 10:24-29, 1994; Tsan et al, Am J Physiol 268; Alton et al., Nat. Genet. 5:135-142, 1993 and U.S. Pat. No. 5,679,647 by Carson et al.

In an exemplary embodiment, the methods and compositions described herein may be used in a variety of applications in plants. For example, it may be desirable to produce a synthetic plant genome that has been modified for purposes of crop development and may be suitable for expressing one or more artificial polypeptides without concern that such traits may spread beyond a controlled environment. For example, modifications of interest may be reflective of the commercial markets and interests of those involved in the development of a crop, including, for example, genes encoding agronomic traits, insect resistance, disease resistance, herbicide resistance, sterility, grain characteristics, commercial products, genes involved in oil, starch, carbohydrate, or nutrient metabolism, genes affecting kernel size, sucrose loading, and the like, and genes involved in grain quality such as levels and types of oils, saturated and unsaturated, quality and quantity of essential amino acids, and levels of cellulose.

Plants and plant cells may be transformed using any method known in the art. In one embodiment, Agrobacterium is employed to introduce a DNA construct into plants. Such transformation typically uses binary Agrobacterium T-DNA vectors (Bevan, 1984, Nuc. Acid Res. 12:8711-8721), and the co-cultivation procedure (Horsch et al., 1985, Science 227:1229-1231). Generally, the Agrobacterium transformation system is used to engineer dicotyledonous plants (Bevan et al., 1982, Ann. Rev. Genet. 16:357-384; Rogers et al., 1986, Methods Enzymol. 118:627-641). The Agrobacterium transformation system may also be used to transform, as well as transfer, DNA to monocotyledonous plants and plant cells. (see Hernalsteen et al., 1984, EMBO J. 3:3039-3041; Hooykaas-Van Slogteren et al., 1984, Nature 311:763-764; Grimsley et al., 1987, Nature 325:1677-179; Boulton et al., 1989, Plant Mol. Biol. 12:31-40; and Gould et al., 1991, Plant Physiol. 95:426-434).

In other embodiments, various alternative methods for introducing recombinant nucleic acid constructs into plants and plant cells may also be utilized. These other methods are particularly useful where the target is a monocotyledonous plant or plant cell. Alternative gene transfer and transformation methods include, but are not limited to, particle gun bombardment (biolistics), protoplast transformation through calcium-, polyethylene glycol (PEG)- or electroporation-mediated uptake of naked DNA (see Paszkowski et al., 1984, EMBO J. 3:2717-2722, Potrykus et al., 1985, Molec. Gen. Genet. 199:169-177; Fromm et al., 1985, Proc. Nat. Acad. Sci. USA 82:5824-5828; and Shimamoto, 1989, Nature 338:274-276) and electroporation of plant tissues (D'Halluin et al., 1992, Plant Cell 4:1495-1505). Additional methods for plant cell transformation include microinjection, silicon carbide mediated DNA uptake (Kaeppler et al., 1990, Plant Cell Reporter 9:415-418), and microprojectile bombardment (see Klein et al., 1988, Proc. Nat. Acad. Sci. USA 85:4305-4309; and Gordon-Kamm et al., 1990, Plant Cell 2:603-618). In various methods, selectable markers may be used, at least initially, in order to determine whether transformation has actually occurred. Useful selectable markers include enzymes which confer resistance to an antibiotic, such as gentamycin, hygromycin, kanamycin and the like. Alternatively, markers which provide a compound identifiable by a color change, such as GUS, or luminescence, such as luciferase, may be used. For plastid transformation, biolistics according the method of Svab and Maliga (Svab et al., 1993, Proc. Natl. Acad. Sci. USA 90: 913-917) is preferred.

The methods and compositions described herein may be practiced with any plant. Such plants include but are not limited to, monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis, tobacco).

The practice of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EQUIVALENTS

The present invention provides among other things methods for assembling large polynucleotide constructs and organisms having increased genomic stability. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications, patents and sequence database entries mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference are the following: U.S. Patent Publication Nos. 2005/0009049; 2003/010885; and 2003/0082575; and Heeb S, et al. Mol Plant Microbe Interact. (2000) 13(2): 232-7; Waters V L Nat. Genet. (2001) 29(4): 375-6; Guiney D G, et al. Plasmid. (1988) 20(3): 259-65; Posfai G, et al. Nucleic Acids Res. (1999) 27(22): 4409-15; Li M Z and Elledge S J. Nat. Genet. (2005) 37(3): 311-9; and Chevalier B S, et al. Mol. Cell. (2002) 10(4): 895-905.

1. A method of expressing an artificial polypeptide comprising at least one unnatural amino acid, the method comprising:
exposing a host cell to an expression condition wherein a first modified tRNA charged with a first unnatural amino acid is available for protein synthesis, wherein the host cell comprises:
a) a genome that is modified to replace at least a threshold number of copies of a first codon with one or more first alternative codons, wherein the first modified tRNA recognizes the first codon with greater specificity than any of the one or more first alternative codons; and
b) a first nucleic acid comprising an open reading frame having at least one copy of the first codon,
wherein translation of the open reading frame results in expression of an artificial polypeptide comprising at least one unnatural amino acid.

2.-4. (canceled)

5. The method of claim 1, wherein at least half of the first codons on the genome are replaced with one or more alternative codons.

6.-11. (canceled)

11. The method of claim 1, wherein the first codon is a first stop codon that is recognized by the first modified tRNA, and the open reading frame is terminated by a stop codon that is different from the first stop codon.

12.-47. (canceled)

48. The method of claim 1, wherein the first unnatural amino acid forms a covalent bond with a second amino acid inside the host cell.

49.-57. (canceled)

58. A crude cell lysate comprising an artificial polypeptide expressed according to the method of claim 1.

59.-61. (canceled)

62. A cell culture supernatant comprising an artificial polypeptide expressed according to the method of claim 1.

63.-65. (canceled)

66. A host cell comprising:
a) a genome that is modified to replace at least a threshold number of copies of a first codon with one or more first alternative codons; and
b) a first nucleic acid that comprises an open reading frame encoding an artificial polypeptide, wherein the open reading frame comprises at least one copy of the first codon, and wherein the first codon is recognized by a first modified tRNA with greater specificity than any of the one or more alternative codons.

67. (canceled)

68. The host cell of claim 66, wherein the first codon is a first stop codon.

69.-87. (canceled)

88. An artificial polypeptide containing at least one unnatural amino acid, wherein the artificial polypeptide was isolated from the crude cell lysate of claim 58.

89. An artificial polypeptide containing at least one unnatural amino acid, wherein the artificial polypeptide was isolated from the cell culture supernatant of claim 62.

90.-100. (canceled)

101. A pharmaceutical preparation comprising an artificial polypeptide isolated from the host cell of claim 66 and a pharmaceutically acceptable excipient, diluent, or carrier.

102.-104. (canceled)

105. The artificial polypeptide of claim 87, wherein the artificial polypeptide comprises a covalent bond between a side chain of the first unnatural amino acid and a side chain of a second amino acid in the artificial polypeptide.

106.-108. (canceled)

109. The artificial polypeptide of claim 105, wherein the covalent bond is internal to a folded structure of the artificial polypeptide.

110.-112. (canceled)

113. An artificial protein comprising an intramolecular covalent bond between a side chain of a first unnatural amino acid and a side chain of a second amino acid, wherein the covalent bond is an internal bond in a folded form of the artificial protein.

114. The artificial protein of claim 113, wherein the second amino acid is unnatural.

115. The artificial protein of claim 114, wherein the first unnatural amino acid is different from the second unnatural amino acid.

116.-120. (canceled)

121. The artificial protein of claim 113, comprising at least two different artificial amino acids.

122. The artificial protein of claim 121, wherein each unnatural amino acid has a side chain that comprises a bioorthogonal functional group.

123.-125. (canceled)

126. The artificial protein of claim 113, wherein the covalent bond thermodynamically stabilizes a folded structure of the artificial protein relative to a corresponding folded structure of a natural or recombinant protein that contains only natural amino acids.

127. The artificial protein of claim 126, wherein the folded structure is a biologically active form of the artificial protein.

128.-176. (canceled)
